(19) United States
(12) Patent Application Publication
(10) Pub. No.: US 2023/0416800 A1
(43) Pub. Date: Dec. 28, 2023

(54) ENZYMATIC METHODS FOR CONVERTING LCA AND 3-KCA TO UDCA AND 3-KUDCA

(71) Applicant: Sandhill One, LLC, West Palm Beach, FL (US)

(72) Inventors: J. GREGORY REID, (US); JAYACHANDRA P. REDDY, West Palm Beach, FL (US); MATT GREGORY, Cambridge (GB); URSULA SCHELL, Cambridge (GB); BERNHARD J. PAUL, Arlington, MA (US)

(21) Appl. No.: 18/038,203

(22) PCT Filed: Nov. 29, 2021
(86) PCT No.: PCT/US2021/061025

§ 371 Date: May 22, 2023

Related U.S. Application Data
(60) Provisional application No. 63/119,188, filed on Nov. 30, 2020.

(51) Int. Cl. C12P 33/06; C12N 15/81; C12N 9/02

(52) U.S. Cl. CPC C12P 33/06; C12N 15/81; C12N 9/0071; C12N 9/0042; C12Y 106/02004; C12Y 114/14001; C12R 2001/84

(57) ABSTRACT

7β-hydroxylation systems are provided, as well as methods for producing ?P-hydroxy derivatives of lithocholic acid and 3-keto-lithocholic acid from such systems. Also provided are recombinant organisms useful for the production of such enzymatic systems, and to plasmids that encode for such enzymes.

### FIELD OF THE INVENTION

The present invention relates to 7β-hydroxylation systems, and to methods for producing 7β-hydroxy derivatives of lithocholic acid and 3-keto-5β-cholanic acid from such systems. The invention also relates to recombinant organisms useful for the production of such enzymatic systems, and to plasmids that encode for such enzymes.

### BACKGROUND OF THE INVENTION

Ursodeoxycholic acid (UDCA) is a valuable bile acid frequently prescribed for the treatment of cholecystitis as it can solubilize cholesterol gallstones with fewer side effects than chenodeoxycholic acid (CDCA). UDCA also has anti-inflammatory properties and is applied in the therapy of cystic fibrosis and liver diseases like primary biliary cholangitis. The major natural source of UDCA is bile from various species of bears.

UDCA can also be produced from cholic acid (CA) or CDCA, also obtained from animal bile. Eggert et al. (2014) report a synthesis route starting from CA to form CDCA in 5 steps, including a Wolff-Kishner ketone reduction, and an epimerization at C7 to produce UDCA. T. Eggert, D. Bakonyi, W. Hummel, J. Biotechnol. 2014, 191, 11-21. Zheng et al. (2015) report a shorter synthesis route based on the biocatalytic epimerization of CDCA to UDCA. M.-M. Zheng, R.-F. Wang, C.-X. Li, J.-H. Xu, Process Biochem. 2015, 50, 598-604.

The association of 7β-hydroxylase systems with cellular membranes is a particular challenge for biocatalytic systems. Indeed, Durairaj et al. (2016) report that P450nor is the only soluble fungal CYP discovered so far, and it performs denitrification. Durairaj et al. Microb Cell Fact (2016) 15:125. The effort is further complicated in whole cell fungi such as Fusarium equiseti wherein, Grobe et al. (2020) reports, the action of multiple P450 enzymes results in side-product formation. S. Grobe, C. Badenhorst, T. Bayer, et al., Angew. Chem. Int. Ed. 10.1002/anie 0.202012675.

To overcome these hurdles, Grobe et al. (2020) report the formation of UDCA from LCA using a variant of cyt P450 monooxygenase CYP107D1 (oleP) from Streptomyces antibioticus, a P450 enzyme that does not require association with a cellular membrane, in an Escherichia coli-based whole-cell system. By modifying the native enzyme, which converts LCA to its 6β-hydroxy derivative, MDCA, the authors were able to mostly change the position of hydroxylation so that UDCA was formed in preference to MDCA. However, the conversion was carried out with very low productivity (at best 67 μM in 24 hr) and with incomplete regioselectivity (at best a ratio of 73:27 of UDCA:MDCA).

A need thus exists for an efficient and productive method for selectively converting LCA and 3-KCA to UDCA and 3-KUDCA. An ideal method would give high yields, be easy to scale-up, and be easy to implement in a commercial production. What is needed are efficient enzymatic systems, processes, and components for the 7β-hydroxylation of lithocholic acid or 3-KCA in commercial volumes.

### SUMMARY OF THE INVENTION

After extensive experimentation with various engineered microbial systems for hydroxylating LCA and 3-KCA, including a series of experiments with yeast transformed with native 7β-hydroxylation systems from other species, the inventors have unexpectedly discovered yeast-based systems transformed to express 7β-hydroxylase activity, that are capable of selectively producing UDCA and 3-KUDCA, and derivatives thereof, from LCA and 3-KCA and derivatives thereof. Thus, in a first principal embodiment the invention provides a method of converting LCA or 3-KCA, or a carboxylic acid ester, carboxylic amide, or carboxylate salt thereof, to UDCA or 3-KUDCA, or a carboxylic acid ester, carboxylic amide, or carboxylate salt thereof, comprising contacting the LCA or 3-KCA, or carboxylic acid ester, carboxylic amide, or carboxylate salt thereof, with a 7β-hydroxylase system in the presence of a yeast, or an extract or lysate thereof, wherein the 7β-hydroxylase system is not native to the yeast.

Further principal embodiments relate to the plasmids used to produce the organisms of the present invention. Thus, in a second principal embodiment the invention provides a plasmid comprising a nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; or SEQ ID NO. 32; or a nucleic acid sequence having at least 85%, 90%, 95%, 98%, or 99%, identity with any of the foregoing sequences.

Additional embodiments relate to the transformed organisms used in the methods of the current invention. Thus, in a third principal embodiment the invention provides an organism transformed by a CYP encoding nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; and SEQ ID NO. 32; or a nucleic acid sequence having at least 85%, 90%, 95%, 98%, or 99%, identity with any of the foregoing nucleic acid sequences.

Still further embodiments relate to the reaction mixture in which the conversions of the present invention take place. Thus, in a fourth principal embodiment the invention provides a reaction mixture comprising (i) LCA or 3-KCA, (ii) a yeast, or an extract or lysate thereof, (iii) a 7β-hydroxylation system. A fifth principal embodiment provides a reaction mixture comprising a yeast and a 7β-hydroxylation system comprising a P450 oxidoreductase (“CPR”) enzyme and a P450 7β-hydroxylase (“CYP”) enzyme, wherein the CYP enzyme is an enzyme native to Gibberella zeae, preferably Gibberella zeae PH1 or Gibberella zeae VKM2600, most preferably Gibberella zeae VKM2600.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 depicts LCMS chromatograms from the experiment described in Example 17. FIG. 1A is a TIC trace of the extracted broth sample. FIG. 1B is a TIC trace of the LCA standard. FIG. 1C is a TIC trace of the UDCA standard.

FIG. 2 is a comparison of the MS spectra for UDCA extracted from broth sample (A) and the UDCA authentic standard (B) reported in example 17.

FIG. 3 depicts CMS chromatograms from the experiment described in Example 18. FIG. 3A is a TIC trace of the isolated UDCA. FIG. 3B is a TIC trace of the UDCA standard.

FIG. 4 is a comparison of the MS spectra for the isolated UDCA (A) and the UDCA authentic standard (B) reported in example 18.

FIG. 5 depicts a ¹H NMR spectrum of isolated UDCA from the experiment described in Example 18.

FIG. 6 depicts a ¹³C NMR spectrum of isolated UDCA from the experiment described in Example 18.

FIG. 7 depicts an ¹H NMR spectrum of authentic UDCA from the experiment described in Example 18.

FIG. 8 depicts a ¹³C NMR spectrum of authentic UDCA from the experiment described in Example 18.

FIG. 9 depicts LCMS chromatograms from the experiment described in Example 19. FIG. 9A is a TIC trace of the extracted broth sample. FIG. 9B is an Extracted Ion Chromatogram (EIC) for m/z 389.3 (3-KUDCA) of the extracted broth sample. FIG. 9C is a TIC trace of the 3-KUDCA standard. FIG. 9D is a TIC trace of the 3-KCA standard.

FIG. 10 is a comparison of the MS spectra for 3-KUDCA extracted from broth sample (A) and the 3-KUDCA authentic standard (B) reported in example 19.

FIG. 11 depicts LCMS chromatograms from the experiment described in Example 21. FIG. 11A is a TIC trace of the extracted broth sample. FIG. 11B is an Extracted Ion Chromatogram (EIC) for m/z 391.3 (UDCA) of the extracted broth sample. FIG. 11C is a TIC trace of the UDCA standard.

FIG. 12 is a comparison of the MS spectra for UDCA extracted from broth sample (A) and the UDCA authentic standard (B), as reported in example 21.

### DETAILED DESCRIPTION

### Definitions and Use of Terms

As used in this specification and in the claims which follow, the singular forms “a,” “an” and “the” include plural referents unless the context clearly dictates otherwise.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality of components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or specifying particular numerical values, it will be understood that a range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

When used herein the term “about” will compensate for variability allowed for in the chemical industry and inherent in products in this industry, such as differences in product strength due to manufacturing variation and time-induced product degradation. In one embodiment, the term allows for ±5% variability or ±10% variability.

The phrase “acceptable” as used in connection with compositions of the invention, refers to molecular entities and other ingredients of such compositions that are physiologically tolerable and do not typically produce untoward reactions when administered to a subject (e.g., a mammal such as a human).

“Coding sequence” refers to that portion of a nucleic acid (e.g., a gene) that encodes an amino acid sequence of a protein.

“Naturally-occurring” or “wild-type” or “native,” in contrast to “non-naturally occurring,” “non-wild-type,” “non-native,” or “foreign,” refers to the form found in nature. For example, a naturally occurring or wild-type polypeptide or polynucleotide sequence is a sequence present in an organism that can be isolated from a source in nature and which has not been intentionally modified by human manipulation.

“Recombinant” when used with reference to, e.g., a cell, nucleic acid, or polypeptide, refers to a material, or a material corresponding to the natural or native form of the material, that has been modified in a manner that would not otherwise exist in nature. Non-limiting examples include, among others, recombinant cells expressing genes that are not found within the native (non-recombinant) form of the cell or express native genes that are otherwise expressed at a different level.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window will comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Those of skill in the art will appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, 150, or 200 or longer windows.

“Substantial identity” refers to a polynucleotide or polypeptide sequence that has at least 80 percent sequence identity, at least 85 percent sequence identity, at least 90% sequence identity, or at least 95 percent sequence identity, more usually at least 98 or 99% sequence identity as compared to a reference sequence over a comparison window comprising at least 90%, 95%, 98%, or 99% of the reference sequence. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

When reference to a cellular organism is given herein, it will be understood to refer to the organism in both its wild-type state and as a modified organism. The term yeast thus includes all of the wild-type yeast existing naturally in nature, in addition to any man-made yeast produced using recombinant techniques.

The term “yeast” refers to Ascomycota Fungi in the Saccaromycetes class, preferably in the Saccharomycetales order, preferably in the Saccharomycetaceae family. Particularly preferred yeasts belong to the Pichia and Saccharomyces genera, especially Pichia pastoris and Saccharomyces cerevisiae.

3-KCA, or 3-keto-5β-cholanic acid is represented by the following chemical structure:

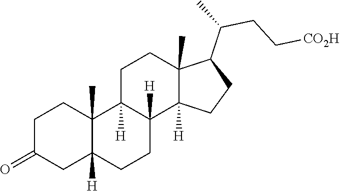

LCA, or lithocholic acid, is represented by the following chemical structure:

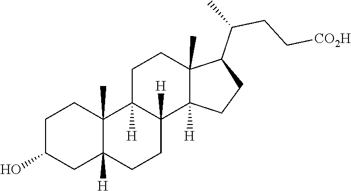

3-KUDCA, or 7β-hydroxy-3-keto-5β-cholanic acid, is represented by the following chemical structure:

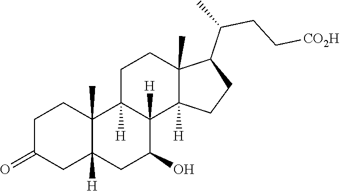

UDCA, or ursodeoxycholic acid, is represented by the following chemical structure:

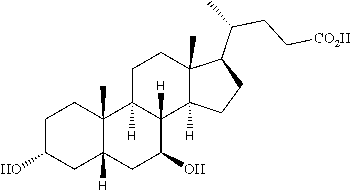

As used herein, carboxylate “salts” refers to derivatives of the disclosed compounds wherein the parent compound is modified by converting an existing acid moiety to its salt form. Examples of suitable salts include, but are not limited to, alkali or organic salts of acidic residues of carboxylic acids. The salts of the present invention include the conventional non-toxic salts or the quaternary ammonium salts of the parent compound formed, for example, from non-toxic inorganic or organic bases. The salts of the present invention can be synthesized from the parent compound which contains an acidic moiety by conventional chemical methods. Generally, such salts can be prepared by reacting the free acid form of these compounds with a stoichiometric amount of the appropriate base in water or in an organic solvent, or in a mixture of the two.

As used herein, an “ester” preferably refers to a —COOR moiety, wherein R is optionally substituted C₁₋₂₀ alkyl, or optionally substituted aryl.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. In any of the embodiments or subembodiments of the present invention, an alkyl group can contain from 1 to about 20, from 2 to about 20, from 1 to about 10, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons (including heteroaromatic hydrocarbons) such as, for example, phenyl, naphthyl, anthracenyl, phenanthrenyl, indanyl, indenyl, and the like. In some embodiments, aryl groups have from 6 to about 20 carbon atoms.

In any of the embodiments or subembodiments of this invention, a moiety which is optionally substituted may be alternatively defined as substituted with 0, 1, 2, or 3 substituents independently selected from halo, OH, amine, C₁₋₆ alkyl, C₁₋₆ alkoxy, C₁₋₆ hydroxyalkyl, CO(C₁₋₆ alkyl), CHO, CO₂H, CO₂(C₁₋₆ alkyl), and C₁₋₆ haloalkyl.

As used herein, an amide preferably refers to a —C(O)N(R′)(R″) moiety, wherein R′ and R″ are independently optionally substituted C₁₋₂₀ alkyl, or optionally substituted aryl. Alternatively, the carboxylic amide of UDCA can be tauroursodeoxycholic acid (“TUDCA”).

The “P450 7β-hydroxylase systems” of the current invention refer to Class II CYP enzyme systems capable of hydroxylating the 7-H position of LCA or K-LCA. As discussed in Durairaj et al. Microb Cell Fact (2016) 15:125, Class II CYP enzyme systems comprise two integral membrane proteins: P450 7β-hydroxylase (referred to herein sometimes as “CYP”) and cytochrome P450 reductase (referred to herein sometimes as “CPR”) containing the prosthetic cofactors FAD and FMN, which deliver two electrons from NAD(P)H to the heme moiety. The system may also comprise a third protein component, Cyt b5, which transfers a second electron to the oxyferrous CYP.

### Discussion of Principal Embodiments

A first principal embodiment of the invention provides a method of converting LCA or 3-KCA, or a carboxylic acid ester, carboxylic amide, or carboxylate salt thereof, to UDCA or 3-KUDCA, or a carboxylic acid ester, carboxylic amide, or carboxylate salt thereof, comprising contacting the LCA or 3-KCA, or carboxylic acid ester, carboxylic amide, or carboxylate salt thereof, with a 7β-hydroxylase system in the presence of a yeast, or an extract or lysate thereof, wherein the 7β-hydroxylase system is not native to the yeast.

A second principal embodiment provides a plasmid comprising a nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; or SEQ ID NO. 32; or a nucleic acid sequence having at least 85%, 90%, 95%, 98%, or 99%, identity with any of the foregoing sequences.

A third principal embodiment provides an organism transformed by a CYP encoding nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; and SEQ ID NO. 32; or a nucleic acid sequence having at least 85%, 90%, 95%, 98%, or 99%, identity with any of the foregoing nucleic acid sequences.

In a fourth principal embodiment the invention provides a reaction mixture comprising (i) LCA or 3-KCA, (ii) a yeast, or an extract or lysate thereof, and (iii) a 7β-hydroxylation system.

A fifth principal embodiment provides a reaction mixture comprising a yeast and a 7β-hydroxylation system comprising a P450 oxidoreductase (“CPR”) enzyme and a P450 7β-hydroxylase (“CYP”) enzyme, wherein the CYP enzyme is an enzyme native to Gibberella zeae, preferably Gibberella zeae PH1 or Gibberella zeae VKM2600, most preferably Gibberella zeae VKM2600.

### Discussion of Subembodiments

As noted previously, the invention is preferably carried out in the presence of a yeast transformed to express a non-native 7β-hydroxylation system. The yeast is preferably selected from Saccharomyces and Pichia, and most preferably is selected from Saccharomyces cerevisiae and Pichia pastoris.

The organisms used in the methods of the current invention will be transformed by a non-native 7β-hydroxylation system comprising a non-native P450 7-beta-hydroxylase (“CYP”) enzyme and optionally a non-native P450 oxidoreductase (“CPR”) enzyme. While the CPR enzyme is critical to the 7β-hydroxylase system, it may not be absolutely necessary that the CPR enzyme be foreign to the organism, as an intrinsic one native to the yeast could be sufficient.

Preferred CYP enzymes for practicing the current invention are encoded by a CYP encoding nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; and SEQ ID NO. 32; or a nucleic acid sequence having at least 85% 90%, 95%, 98%, or 99%, identity with any of the foregoing nucleic acid sequences.

The nucleic acid encoding the CYP can be selected from any one or combination of the foregoing SEQ ID NOs, and combined with any of the CPR enzymes of the current invention. In one embodiment the encoding nucleic acid sequence is selected from SEQ ID NO. 8; SEQ ID NO. 11; SEQ ID NO. 14; SEQ ID NO. 17; and SEQ ID NO. 20; or a nucleic acid sequence having at least 85%, 90%, 95%, 98%, or 99%, identity with any of the foregoing sequences. In another embodiment the nucleic acid is selected from SEQ ID NO. 23; SEQ ID NO. 26; or SEQ ID NO. 29; or a nucleic acid sequence having at least 85%, 90%, 95%, 98%, or 99%, identity with any of the foregoing sequences. In still another embodiment the nucleic acid sequence is selected from SEQ ID NO. 32; or a nucleic acid sequence having at least 85%, 90%, 95%, 98%, or 99%, identity with SEQ ID NO. 32.

The CYP enzyme preferably comprises a CYP amino acid sequence selected from SEQ ID NO. 9; SEQ ID NO. 12; SEQ ID NO. 15; SEQ ID NO. 18; SEQ ID NO. 21; SEQ ID NO. 24; SEQ ID NO. 27; SEQ ID NO. 30; or SEQ ID NO. 33; or an amino acid sequence having at least 85% 90%, 95%, 98%, or 99%, identity with any of the foregoing amino acid sequences.

The CYP enzyme can be selected from any one or combination of the foregoing SEQ ID NOs, and combined with any of the CPR enzymes of the current invention. In one embodiment the CYP enzyme comprises SEQ ID NO. 9; SEQ ID NO. 12; SEQ ID NO. 15; SEQ ID NO. 18; and SEQ ID NO. 21; or an amino acid sequence having at least 85%, 90%, 95%, 98%, or 99%, identity with any of the foregoing sequences. In another embodiment the CYP enzyme comprises SEQ ID NO. 24; SEQ ID NO. 27; or SEQ ID NO. 30; or an amino acid sequence having at least 85%, 90%, 95%, 98%, or 99%, identity with any of the foregoing sequences. In still another embodiment the CYP enzyme comprises SEQ ID NO. 33; or an amino acid sequence having at least 85%, 90%, 95%, 98%, or 99%, identity with SEQ ID NO. 33.

Preferred plasmids encoding the CYP enzymes of the current invention preferably comprise a nucleic acid sequence selected from SEQ ID NO. 7; SEQ ID NO. 10; SEQ ID NO. 13; SEQ ID NO. 16; SEQ ID NO. 19; SEQ ID NO. 22; SEQ ID NO. 25; SEQ ID NO. 28; or SEQ ID NO. 31; or a nucleic acid sequence having at least 85% 90%, 95%, 98%, or 99%, identity with any of the foregoing nucleic acid sequences.

In one embodiment the plasmid encoding the CYP enzyme comprises SEQ ID NO. 7; SEQ ID NO. 10; SEQ ID NO. 13; SEQ ID NO. 16; or SEQ ID NO. 19; or a nucleic acid sequence having at least 85%, 90%, 95%, 98%, or 99%, identity with any of the foregoing sequences. In another embodiment the plasmid encoding the CYP enzyme comprises SEQ ID NO. 22; SEQ ID NO. 25; or SEQ ID NO. 28; or a nucleic acid sequence having at least 85%, 90%, 95%, 98%, or 99%, identity with any of the foregoing sequences. In still another embodiment the plasmid encoding the CYP enzyme comprises SEQ ID NO. 31; or a nucleic acid sequence having at least 85%, 90%, 95%, 98%, or 99%, identity with SEQ ID NO. 31.

In one embodiment, the CYP enzyme is a protein native to Gibberella zeae, preferably Gibberella zeae PH1 or Gibberella zeae VKM2600, most preferably Gibberella zeae VKM2600, and the organism is transformed to express such protein.

The CPR enzyme in the 7β-hydroxylation system can be native to the organism in which the 7β-hydroxylase activity is expressed, or encoded by a CPR encoding nucleic acid sequence selected from SEQ ID NO. 2 and SEQ ID NO. 5, or a nucleic acid sequence having at least 85% 90%, 95%, 98%, or 99%, identity with any of the foregoing nucleic acid sequences. The CPR enzyme preferably comprises a CPR amino acid sequence selected from SEQ ID NO. 3 and SEQ ID NO. 6, or an amino acid sequence having at least 85% 90%, 95%, 98%, or 99%, identity with any of the foregoing amino acid sequences.

In one embodiment the methods of the current invention are practiced to produce UDCA or a carboxylic acid ester, carboxylic amide, or carboxylate salt thereof, by contacting LCA, or a carboxylic acid ester, carboxylic amide, or carboxylate salt thereof, with the 7β-hydroxylase system. In another embodiment the methods of the current invention are practiced by contacting 3-KCA or a carboxylic acid ester, carboxylic amide, or carboxylate salt thereof, with the 7β-hydroxylase system to produce 3-KUDCA or a carboxylic acid ester, carboxylic amide, or carboxylate salt thereof. When 3-KUDCA or a carboxylic acid ester, carboxylic amide, or carboxylate salt thereof, is produced, the methods of the current invention will optionally further comprise reducing the 3-KUDCA or carboxylic acid ester, carboxylic amide, or carboxylate salt thereof to UDCA or a carboxylic acid ester, carboxylic amide, or carboxylate salt thereof.

In preferred embodiments, the methods of the current invention further comprise isolating the UDCA or 3-KUDCA, or carboxylic acid ester, carboxylic amide, or carboxylate salt thereof, from the 7β-hydroxylase system. By isolated it is meant that the UDCA or 3-KUDCA is substantially pure of the 7β-hydroxylase system and the reaction mixture in which the UDCA or 3-KUDCA was produced. Thus, the UDCA or 3-KUDCA is at least 90% pure, at least 95% pure, or at least 98% pure, when the weight of any residual reaction mixture is considered. In particularly preferred embodiments, the UDCA or 3-KUDCA, or carboxylic acid ester, carboxylic amide, or carboxylate salt thereof, is produced substantially as a pure diastereoisomer. A “substantially pure diastereomer” refers to a diastereomer that is at least 90% pure, at least 95% pure, or at least 98% pure, when its 7α-diastereomer is considered.

### Engineered CYP and CPR Enzymes

CYP and CPR enzymes having different properties than the enzyme sequences disclosed herein can be obtained by mutating the genetic material encoding the CYP or CPR enzyme and identifying polynucleotides that express engineered enzymes with a desired property. These non-naturally occurring CYP and CPR enzymes can be generated by various well-known techniques, such as in vitro mutagenesis or directed evolution. In some embodiments, directed evolution is an attractive method for generating engineered enzymes because of the relative ease of generating mutations throughout the whole of the gene coding for the polypeptide, as well as providing the ability to take previously mutated polynucleotides and subjecting them to additional cycles of mutagenesis and/or recombination to obtain further improvements in a selected enzyme property. Subjecting the whole gene to mutagenesis can reduce the bias that may result from restricting the changes to a limited region of the gene. It can also enhance generation of enzymes affected in different enzyme properties since distantly spaced parts of the enzyme may play a role in various aspects of enzyme function.

In mutagenesis and directed evolution, the parent or reference polynucleotide encoding the naturally occurring or wild type CYP or CPR enzyme is subjected to mutagenic processes, for example random mutagenesis and recombination, to introduce mutations into the polynucleotide. The mutated polynucleotide is expressed and translated, thereby generating engineered CYP or CPR enzymes with modifications to the polypeptide. As used herein, “modifications” include amino acid substitutions, deletions, and insertions. Any one or a combination of modifications can be introduced into the naturally occurring enzymatically active polypeptide to generate engineered enzymes, which are then screened by various methods to identify polypeptides, and corresponding polynucleotides, having a desired improvement in a specific enzyme property.

### 7-Beta Hydroxylase Environment

The CYP and CPR enzymes may be present within a cell, in the cellular medium, on an immobilized substrate, or in other forms, such as lysates and extracts of cells recombinantly designed to express the enzyme, or isolated preparations. The term “isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

In some embodiments, the isolated CYP and CPR enzymes are present in a substantially pure polypeptide composition. The term “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure CYP and CPR enzyme composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of singular CYP and CPR macromolecular species. Solvent species, small molecules (<500 Daltons), and elemental ion species are not considered macromolecular species.

### Encoding Polynucleotide

An isolated polynucleotide encoding a CYP and CPR enzyme may be manipulated in a variety of ways to provide for expression of the enzyme. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006.

Thus, in another aspect, the present disclosure is also directed to a recombinant expression vector comprising a polynucleotide encoding a CYP and CPR enzyme polypeptide or a variant thereof, and one or more expression regulating regions such as a promoter and a terminator, a replication origin, etc., depending on the type of hosts into which they are to be introduced. The various nucleic acid and control sequences may be joined together to produce a recombinant expression vector which may include one or more convenient restriction sites to allow for insertion or substitution of the nucleic acid sequence encoding the polypeptide at such sites. In creating the recombinant expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell may be used. In particularly preferred embodiments, the plasmids or vectors of the current invention are under control of an AOX1 promoter and an AOX1 terminator sequence.

The term “control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, pro-peptide sequence, promoter, signal peptide sequence, and transcription terminator. At a minimum, the control sequences include a promoter, transcriptional and translational stop signals, and a ribosome binding site (to stop translation). The control sequences may be provided with linkers for the purpose of introducing specific restriction sites facilitating ligation of the control sequences with the coding region of the nucleic acid sequence encoding a polypeptide.

The term “operably linked” is defined herein is a configuration in which a control sequence is appropriately placed at a position relative to the coding sequence of the DNA sequence such that the control sequence directs the expression of a polynucleotide and/or polypeptide. The control sequence may be an appropriate promoter sequence. The “promoter sequence” is a nucleic acid sequence that is recognized by a host cell for expression of the coding region. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

### Host Cells for Expression of CYP and CPR Polypeptides

In another aspect, the present disclosure provides a host cell comprising polynucleotides encoding CYP and CPR enzymes of the present disclosure, the polynucleotides being operatively linked to one or more control sequences for expression of the CYP and CPR enzymes in the host cell. Host cells for use in expressing the CYP and CPR enzymes encoded by the expression vectors of the present invention are well known in the art and include particularly the yeast cells of the current invention (e.g., Saccharomyces cerevisiae or Pichia pastoris). In one particular embodiment, the process of the current invention is carried out with whole cells that express the CYP and CPR enzyme, or an extract or lysate of such cells, wherein the whole cells or extract or lysate of such whole cells are selected from Pichia pastoris and Saccharomyces cerevisiae. Appropriate culture mediums and growth conditions for the above-described host cells are well known in the art.

Polynucleotides for expression of the CYP and CPR enzyme may be introduced into cells by various methods known in the art. For the yeasts described herein, the typical process is by transformation (e.g. electroporation or calcium chloride mediated) or conjugation, or sometimes protoplast fusion. Various methods for introducing polynucleotides into cells will be apparent to the skilled artisan.

### Reaction Conditions

In carrying out the stereoselective hydroxylation described herein, the CYP and CPR enzyme may be added to the reaction mixture in the form of the purified enzymes (including immobilized variants), whole cells transformed with gene(s) encoding the enzymes, and/or cell extracts and/or lysates of such cells. The gene(s) encoding the engineered CYP and CPR enzyme can be transformed into host cells separately or together into the same host cell.

For example, in some embodiments one set of host cells can be transformed with gene(s) encoding the CYP enzyme and another set can be transformed with gene(s) encoding the CPR enzyme. Both sets of transformed cells can be utilized together in the reaction mixture in the form of whole cells, or in the form of lysates or extracts derived therefrom. In other embodiments, a host cell can be transformed with gene(s) encoding both the engineered CYP and CPR enzymes.

Whole cells transformed with gene(s) encoding the CYP and CPR enzymes, or cell extracts and/or lysates thereof, may be employed in a variety of different forms, including solid (e.g., lyophilized, spray-dried, immobilized, and the like) or semisolid (e.g., a crude paste). The cell extracts or cell lysates may be partially purified by precipitation (ammonium sulfate, polyethyleneimine, heat treatment or the like), followed by a desalting procedure prior to lyophilization (e.g., ultrafiltration, dialysis, and the like).

The quantities of reactants used in the hydroxylation reaction will generally vary depending on the quantities of CYP and CPR enzyme substrate employed. The following guidelines can be used to determine the amounts of CYP and CPR enzyme to use. Generally, sterol substrates are employed at a concentration of about 1 to 20 grams/liter using from about 50 mg/liter to about 5 g/liter of the hydroxylase system. The weight ratio of the sterol to the hydroxylase system in the reaction mixture is commonly from about 10:1 to 200:1. Those having ordinary skill in the art will readily understand how to vary these quantities to tailor them to the desired level of productivity and scale of production.

The order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the hydroxylase system may be added first to the solvent. Preferably, however, the enzyme preparation is added last.

Suitable conditions for carrying out the CYP and CPR enzyme-catalyzed reactions described herein include a wide variety of conditions including contacting the CYP and CPR enzymes and sterol substrate at an experimental pH and temperature and detecting product, for example, using the methods described in the Examples provided herein.

The hydroxylase-catalyzed reactions described herein are generally carried out in a solvent. While water is most preferred, organic solvents such as ethyl acetate, butyl acetate, 1-octanol, heptane, octane, methyl t-butyl ether (MTBE), toluene, and the like, and ionic liquids such as 1-ethyl 4-m ethylimi dazolium tetrafluoroborate, 1-butyl-3-methylimidazolium tetrafluoroborate, 1-butyl-3-methylimi dazolium hexafluorophosphate, and the like, can be used in certain circumstances, either alone or in combination with water. In preferred embodiments, aqueous solvents, including water and aqueous co-solvent systems, are used. The solvent system is preferably greater than 50%, 75%, 90%, 95%, or 98% water, and in one embodiment is 100% water.

During the course of the hydroxylation, the pH of the reaction mixture may change. The pH of the reaction mixture may be maintained at a desired pH or within a desired pH range by the addition of an acid or a base during the course of the reaction. Alternatively, the pH may be controlled by using a solvent that comprises a buffer. Suitable buffers to maintain desired pH ranges are known in the art and include, for example, phosphate buffer, triethanolamine buffer, and the like. Combinations of buffering and acid or base addition may also be used.

The hydroxylation is typically carried out at a temperature in the range of from about 15° C. to about 75° C. For some embodiments, the reaction is carried out at a temperature in the range of from about 20° C. to about 55° C. In still other embodiments, it is carried out at a temperature in the range of from about 20° C. to about 45° C. The reaction may also be carried out under ambient conditions.

The reaction is generally allowed to proceed until essentially complete, or near complete, hydroxylation of substrate is obtained. Hydroxylation of substrate to product can be monitored using known methods by detecting substrate and/or product. Suitable methods include gas chromatography, HPLC, and the like. Conversion yields of the sterol hydroxylation product generated in the reaction mixture are generally greater than about 50%, may also be greater than about 60%, may also be greater than about 70%, may also be greater than about 80%, may also be greater than 90%, and can even be greater than about 97%.

The hydroxylation product can be recovered from the reaction mixture and optionally further purified using methods that are known to those of skill in the art. Chromatographic techniques for isolation from the hydroxylase system include, among others, reverse phase chromatography high performance liquid chromatography, ion exchange chromatography, gel electrophoresis, and affinity chromatography. Conditions for purifying a particular sterol will depend, in part, on factors such as net charge, hydrophobicity, hydrophilicity, molecular weight, molecular shape, etc., and will be apparent to those having skill in the art. A preferred method for product purification involves extraction into an organic solvent and subsequent crystallization.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### General Methods for Examples 1-15

Isolation, handling and manipulation of DNA are carried out using standard methods (Green and Sambrook, 2012), which includes digestion with restriction enzymes, PCR, cloning techniques and transformation of bacterial cells. See, e.g., Green, M. R., Sambrook, J., 2012. Molecular Cloning: A Laboratory Manual, Fourth Edition, 4 Lab edition. ed. Cold Spring Harbor Press, Cold Spring Harbor, N.Y.

Synthetic DNA is ordered from a commercial vendor, such as Eurofins Scientific SE (Brussels Belgium), Integrated DNA Technologies (Coralville, Iowa), Genewiz (a Brooks Life Sciences Company) (South Plainfield, New Jersey), or Twist Bioscience (San Francisco, California). Genes are supplied in custom vectors as described in the examples.

### Media

2TY medium contains 16 g/L bacto-tryptone, 10 g/L yeast extract and 5 g/L NaCl and is sterilized by autoclaving. 2TY agar additionally contains 15 g/L agar.

YPD medium contains 10 g/L yeast extract, 10 g/L bacto-tryptone and is sterilized by autoclaving. 50 ml/L of sterile 40% glucose stock solution is added just before use. YPD agar plates additionally contain 15 g/L agar.

BMG contains 100 mM potassium phosphate, pH 7.5, 13.4 g/L YNB, 0.4 mg/L biotin and 1% glycerol.

BMM contains 100 mM potassium phosphate, pH 7.5, 13.4 g/L YNB, 0.4 mg/L biotin and 1% methanol.

BMMY medium is made by dissolving 10 g yeast extract and 10 g bacto-tryptone in 700 ml dH₂O and sterilization by autoclaving. Just before use, 100 ml YNB stock solution, 2 ml biotin stock solution and 100 ml 100 mM potassium phosphate buffer, pH 6.0 are added.

YNB stock solution consists of 134 g/L yeast nitrogen base with ammonium sulfate and without amino acids and is sterilized by autoclaving

Biotin stock solution consists of 200 mg/L biotin and is sterilized by filtration using a 0.2 μm filter.

### Materials

Restriction enzymes are purchased from New England Biolabs (Ipswich, Massachusetts) or Promega Corporation (Madison, Wisconsin). Media components, chemicals and PCR primers are obtained from MilliporeSigma (St. Louis, Missouri). Zeocin is supplied by Thermo Fisher Scientific (Waltham, Massachusetts).

Transformation of Pichia pastoris

Pichia pastoris (Komagataella phaffi NRRL Y-11430/ATCC 76273, hereafter referred to as Pichia pastoris SAND101) is grown overnight in 10 ml YPD at 30° C., shaking at 250 RPM. This culture is used to inoculate 500 ml YPD to an OD600 of 0.1, which is then incubated at 30° C., shaking at 250 RPM to an OD600 of 1.3-1.5. Cells are harvested by centrifugation at 2000×g at 4° C. for 10 minutes and resuspended in 100 ml YPD supplemented with 20 ml 1 M HEPES, pH 8.0 and 2.5 ml 1 M DTT. Cells are incubated at 30° C. without shaking for 15 minutes. Cold dH₂O is added to a final volume of 500 ml and cells are harvested by centrifugation at 2000×g at 4° C. for 10 minutes. Cells are washed with 250 ml cold dH₂O and harvested by centrifugation at 2000 xg at 4° C. for 10 minutes. Cells are washed with 20 ml cold 1 M sorbitol and harvested by centrifugation at 2000×g at 4° C. for 10 minutes. Cells are resuspended in 500 μl cold 1 M sorbitol. 100 ng DNA is added to 40 μl of the competent cells and transferred to a 2 mm gap electroporation cuvette, precooled on ice. Cells are electroporated on a BTRX ECM 630 decay wave electroporation system, using 1500 V, 200Ω, 25 μF settings. 1 ml cold 1 M sorbitol is added immediately, and the mixture is transferred to a sterile Eppendorf tube. Cells are regenerated at shaking at 250 RPM for at least 30 minutes. Cells are then spread onto YPD agar plates containing appropriate antibiotics, then incubated at 30° C. for 2 days or until colonies become visible.

### Example 1: Construction of a Pichia pastoris Strain Capable of Expressing SEQ ID NO. 2 (FGSG_04903)

Plasmid pSAND102 is obtained as synthetic DNA with the sequence SEQ ID NO. 1 from a commercial provider. In brief, it contains the AOX1 promoter sequence, followed by a gene with sequence SEQ ID NO. 2, encoding a P450 reductase with sequence SEQ ID NO. 3, under control of the AOX1 promoter, followed by the AOX1 terminator sequence. The AOX1 promoter contains a unique PmeI restriction site to allow linearization of plasmid pSAND102

Plasmid pSAND102 is linearized with restriction enzyme PmeI. Linearized plasmid is purified from the reaction mixture, e.g. using a commercially available column purification kit. Electrocompetent cells of strain Pichia pastoris SAND101 are transformed with PmeI-linearized plasmid pSAND102, enabling it to integrate into the genome at the AOX1 promoter. Transformants are plated onto YPD agar containing 100 μg/ml nourseothricin and incubated at until colonies become visible. The resulting strain is named Pichia pastoris SAND102.

### Example 2: Construction of a Pichia pastoris Strain Capable of Expressing SEQ ID NO. 5 (FGSG_03175)

Plasmid pSAND103 is obtained as synthetic DNA with the sequence SEQ ID NO. 4 from a commercial provider. In brief, it contains the AOX1 promoter sequence, followed by a gene with sequence SEQ ID NO. 5, encoding a P450 reductase with sequence SEQ ID NO. 6, under control of the AOX1 promoter, followed by the AOX1 terminator sequence. The AOX1 promoter contains a unique PmeI restriction site to allow linearization of plasmid pSAND103

Plasmid pSAND103 is linearized with restriction enzyme PmeI. Linearized plasmid is purified from the reaction mixture, e.g. using a commercially available column purification kit. Electrocompetent cells of strain Pichia pastoris SAND101 are transformed with PmeI-linearized plasmid pSAND103, enabling it to integrate into the genome at the AOX1 promoter. Transformants are plated onto YPD agar containing 100 μg/ml nourseothricin and incubated at 30° C. until colonies become visible. The resulting strain is named Pichia pastoris SAND103.

### Example 3: Construction of Pichia pastoris Strains Capable of Expressing SEQ ID NO. 8 (FGSG_05333)

Plasmid pSAND104 is obtained as synthetic DNA with the sequence SEQ ID NO. 7 from a commercial provider. In brief, it contains the AOX1 promoter sequence, followed by a gene with sequence SEQ ID NO. 8, encoding a P450 with sequence SEQ ID NO. 9, under control of the AOX1 promoter, followed by the AOX1 terminator sequence.

Electrocompetent cells of strain Pichia pastoris SAND102 are transformed with plasmid pSAND104, plated onto YPD agar containing 100 μg/mlnourseothricin and 100 μg/ml zeocin and incubated at 30° C. until colonies become visible. The resulting strain is named Pichia pastoris SAND104.

Electrocompetent cells of strain Pichia pastoris SAND103 are transformed with plasmid pSAND104, plated onto YPD agar containing 100 μg/ml nourseothricin and 100 μg/ml zeocin and incubated at 30° C. until colonies become visible. The resulting strain is named Pichia pastoris SAND105.

### Example 4: Construction of Pichia pastoris Strains Capable of Expressing SEQ ID NO. 11 (FGSG_02672)

Plasmid pSAND105 is obtained as synthetic DNA with the sequence SEQ ID NO. 10 from a commercial provider. In brief, it contains the AOX1 promoter sequence, followed by a gene with sequence SEQ ID NO. 11, encoding a P450 with sequence SEQ ID NO. 12, under control of the AOX1 promoter, followed by the AOX1 terminator sequence.

Electrocompetent cells of strain Pichia pastoris SAND102 are transformed with plasmid pSAND105, plated onto YPD agar containing 100 μg/ml nourseothricin and 100 μg/ml zeocin and incubated at 30° C. until colonies become visible. The resulting strain is named Pichia pastoris SAND106.

Electrocompetent cells of strain Pichia pastoris SAND103 are transformed with plasmid pSAND105, plated onto YPD agar containing 100 μg/ml nourseothricin and 100 μg/ml zeocin and incubated at 30° C. until colonies become visible. The resulting strain is named Pichia pastoris SAND107.

### Example 5: Construction of Pichia pastoris Strains Capable of Expressing SEQ ID NO. 14 (FGSG_10695)

Plasmid pSAND106 is obtained as synthetic DNA with the sequence SEQ ID NO. 13 from a commercial provider. In brief, it contains the AOX1 promoter sequence, followed by a gene with sequence SEQ ID NO. 14, encoding a P450 with sequence SEQ ID NO. 15, under control of the AOX1 promoter, followed by the AOX1 terminator sequence.

Electrocompetent cells of strain Pichia pastoris SAND102 are transformed with plasmid pSAND106, plated onto YPD agar containing 100 μg/ml nourseothricin and 100 μg/ml zeocin and incubated at 30° C. until colonies become visible. The resulting strain is named Pichia pastoris SAND108.

Electrocompetent cells of strain Pichia pastoris SAND103 are transformed with plasmid pSAND106, plated onto YPD agar containing 100 μg/ml nourseothricin and 100 μg/ml zeocin and incubated at 30° C. until colonies become visible. The resulting strain is named Pichia pastoris SAND109.

### Example 6: Construction of Pichia pastoris Strains Capable of Expressing SEQ ID NO. 17 (P450 51(1)—FGSG_04092)

Plasmid pSAND107 is obtained as synthetic DNA with the sequence SEQ ID NO. 16 from a commercial provider. In brief, it contains the AOX1 promoter sequence, followed by a gene with sequence SEQ ID NO. 17, encoding a P450 with sequence SEQ ID NO. 18, under control of the AOX1 promoter, followed by the AOX1 terminator sequence.

Electrocompetent cells of strain Pichia pastoris SAND102 are transformed with plasmid pSAND107, plated onto YPD agar containing 100 μg/ml nourseothricin and 100 μg/ml zeocin and incubated at 30° C. until colonies become visible. The resulting strain is named Pichia pastoris SAND110.

Electrocompetent cells of strain Pichia pastoris SAND103 are transformed with plasmid pSAND107, plated onto YPD agar containing 100 μg/ml nourseothricin and 100 μg/ml zeocin and incubated at 30° C. until colonies become visible. The resulting strain is named Pichia pastoris SAND111.

### Example 7: Construction of Pichia pastoris Strains Capable of Expressing SEQ ID NO. 20 (P450 51(2)—FGSG_01000)

Plasmid pSAND108 is obtained as synthetic DNA with the sequence SEQ ID NO. 19 from a commercial provider. In brief, it contains the AOX1 promoter sequence, followed by a gene with sequence SEQ ID NO. 20, encoding a P450 with sequence SEQ ID NO. 21, under control of the AOX1 promoter, followed by the AOX1 terminator sequence.

Electrocompetent cells of strain Pichia pastoris SAND102 are transformed with plasmid pSAND108, plated onto YPD agar containing 100 μg/ml nourseothricin and 100 μg/ml zeocin and incubated at 30° C. until colonies become visible. The resulting strain is named Pichia pastoris SAND112.

Electrocompetent cells of strain Pichia pastoris SAND103 are transformed with plasmid pSAND108, plated onto YPD agar containing 100 μg/ml nourseothricin and 100 μg/ml zeocin and incubated at 30° C. until colonies become visible. The resulting strain is named Pichia pastoris SAND113.

### Example 8: Construction of Pichia pastoris Strains Capable of Expressing SEQ ID NO. 23 (FGRAMPH1_01T05089)

Plasmid pSAND109 is obtained as synthetic DNA with the sequence SEQ ID NO. 22 from a commercial provider. In brief, it contains the AOX1 promoter sequence, followed by a gene with sequence SEQ ID NO. 23, encoding a P450 with sequence SEQ ID NO. 24, under control of the AOX1 promoter, followed by the AOX1 terminator sequence.

Electrocompetent cells of strain Pichia pastoris SAND102 are transformed with plasmid pSAND109, plated onto YPD agar containing 100 μg/ml nourseothricin and 100 μg/ml zeocin and incubated at 30° C. until colonies become visible. The resulting strain is named Pichia pastoris SAND114.

Electrocompetent cells of strain Pichia pastoris SAND103 are transformed with plasmid pSAND109, plated onto YPD agar containing 100 μg/ml nourseothricin and 100 μg/ml zeocin and incubated at 30° C. until colonies become visible. The resulting strain is named Pichia pastoris SAND115.

### Example 9: Construction of Pichia pastoris Strains Capable of Expressing SEQ ID NO. 26 (FGRAMPH1_01T09325)

Plasmid pSAND110 is obtained as synthetic DNA with the sequence SEQ ID NO. 25 from a commercial provider. In brief, it contains the AOX1 promoter sequence, followed by a gene with sequence SEQ ID NO. 26, encoding a P450 with sequence SEQ ID NO. 27, under control of the AOX1 promoter, followed by the AOX1 terminator sequence.

Electrocompetent cells of strain Pichia pastoris SAND102 are transformed with plasmid pSAND110, plated onto YPD agar containing 100 μg/ml nourseothricin and 100 μg/ml zeocin and incubated at 30° C. until colonies become visible. The resulting strain is named Pichia pastoris SAND116.

Electrocompetent cells of strain Pichia pastoris SAND103 are transformed with plasmid pSAND110, plated onto YPD agar containing 100 μg/ml nourseothricin and 100 μg/ml zeocin and incubated at 30° C. until colonies become visible. The resulting strain is named Pichia pastoris SAND117.

### Example 10: Construction of Pichia pastoris Strains Capable of Expressing SEQ ID NO. 29 (FGRAMPH1_01T21239)

Plasmid pSAND111 is obtained as synthetic DNA with the sequence SEQ ID NO. 28 from a commercial provider. In brief, it contains the AOX1 promoter sequence, followed by a gene with sequence SEQ ID NO. 29, encoding a P450 with sequence SEQ ID NO. 30, under control of the AOX1 promoter, followed by the AOX1 terminator sequence.

Electrocompetent cells of strain Pichia pastoris SAND102 are transformed with plasmid pSAND111, plated onto YPD agar containing 100 μg/ml nourseothricin and 100 μg/ml zeocin and incubated at 30° C. until colonies become visible. The resulting strain is named Pichia pastoris SAND118.

Electrocompetent cells of strain Pichia pastoris SAND103 are transformed with plasmid pSAND111, plated onto YPD agar containing 100 μg/ml nourseothricin and 100 μg/ml zeocin and incubated at 30° C. until colonies become visible. The resulting strain is named Pichia pastoris SAND119.

### Example 11: Construction of Pichia pastoris Strains Capable of Expressing SEQ ID NO. 32 (FGSG_02672V2)

Plasmid pSAND112 is obtained as synthetic DNA with the sequence SEQ ID NO. 31 from a commercial provider. In brief, it contains the AOX1 promoter sequence, followed by a gene with sequence SEQ ID NO. 32, encoding a P450 with sequence SEQ ID NO. 33, under control of the AOX1 promoter, followed by the AOX1 terminator sequence.

Electrocompetent cells of strain Pichia pastoris SAND102 are transformed with plasmid pSAND112, plated onto YPD agar containing 100 μg/ml nourseothricin and 100 μg/ml zeocin and incubated at 30° C. until colonies become visible. The resulting strain is named Pichia pastoris SAND120.

Electrocompetent cells of strain Pichia pastoris SAND103 are transformed with plasmid pSAND112, plated onto YPD agar containing 100 μg/ml nourseothricin and 100 μg/ml zeocin and incubated at 30° C. until colonies become visible. The resulting strain is named Pichia pastoris SAND121.

### Example 12: Expression of P450 and P450 Reductase Genes in Pichia Pastoris Strains Pichia pastoris Sand104—Pichia pastoris Sand121

Conversion of lithocholic acid to ursodeoxycholic acid by strains Pichia pastoris SAND104, Pichia pastoris SAND105, Pichia pastoris SAND106, Pichia pastoris SAND107, Pichia pastoris SAND108, Pichia pastoris SAND109, Pichia pastoris SAND110, Pichia pastoris SAND111, Pichia pastoris SAND112, Pichia pastoris SAND113, Pichia pastoris SAND114, Pichia pastoris SAND115, Pichia pastoris SAND116, Pichia pastoris SAND117, Pichia pastoris SAND118, Pichia pastoris SAND119, Pichia pastoris SAND120, and Pichia pastoris SAND121 is tested by induction of gene expression using standard methods. In one such method, YPD medium, containing 100 μg/ml nourseothricin and 100 μg/ml zeocin, is inoculated with a fresh single colony of the strain and incubated overnight at 30° C. shaking at 250 RPM. Fresh BMMY medium containing 2 mM aminolevulinic acid, 100 μl/ml nourseothricin and 100 μg/ml zeocin is inoculated with 1/10th volume overnight culture and incubated at 30° C. shaking at 250 RPM until an OD600 of 1.0 is reached. Methanol is added to a final concentration of 0.5% (v/v), lithocholic acid is added to a final concentration of 1 mM and incubation is resumed at 30° C. shaking at 250 RPM for 2-3 days.

Products, including UDCA, are extracted from the broth using standard methods, such as those described in X. Ma, and X. Cao, Bioresources and Bioprocessing volume 1, Article number: (2014) and F. Tonin and I. Arends, Beilstein J Org Chem. 2018; 14: 470-483. In one method, the culture is extracted into an equal volume of ethyl acetate and the pH is adjusted to less than 4 by the addition of an acid, the ethyl acetate phase is separated and then the solvent is removed by evaporation, then the sterol of interest is purified using chromatography.

### Example 13: LCA Conversion Using Whole Cells of Pichia pastoris Strains Pichia pastoris Sand104—Pichia pastoris Sand121 Grown on Bmg Medium

Conversion of lithocholic acid to ursodeoxycholic acid by strains Pichia pastoris SAND104, Pichia pastoris SAND105, Pichia pastoris SAND106, Pichia pastoris SAND107, Pichia pastoris SAND108, Pichia pastoris SAND109, Pichia pastoris SAND110, Pichia pastoris SAND111, Pichia pastoris SAND112, Pichia pastoris SAND113, Pichia pastoris SAND114, Pichia pastoris SAND115, Pichia pastoris SAND116, Pichia pastoris SAND117, Pichia pastoris SAND118, Pichia pastoris SAND119, Pichia pastoris SAND120, and Pichia pastoris SAND121 is tested by induction of gene expression using standard methods, such as that described in W. Lu, J. Feng, X. Chen, et al., 2019 Appl. Environ. Microbiol. 85, e01182-19. In this method, 25 ml BMG medium is inoculated with a fresh single colony of the strain and incubated at 30° C. shaking at 250 RPM to an OD600 of 10. Cells are harvested by centrifugation at 4000×g for 5 minutes and suspended in BMM medium containing 2 mM aminolevulenic acid to an OD600 of 1.0. The culture is incubated at 20° C. shaking at 250 RPM with addition of methanol (1% v/v) every 24 hours for 5 days.

Cells are harvested by centrifugation at 4000×g for 5 minutes and resuspended in 30 ml mM potassium phosphate buffer, pH 7.5 containing 2 mM aminolevulinic acid and 1 mM lithocholic acid. The cell suspension is incubated at 30° C. shaking at 200 RPM with addition of methanol (1% v/v) every 24 hours for 3 days.

Products, including UDCA, are extracted from the broth using standard methods, such as those described in X. Ma, and X. Cao, Bioresources and Bioprocessing volume 1, Article number: 5 (2014) and F. Tonin and I. Arends, Beilstein J Org Chem. 2018; 14: 470-483. In one method, the culture is extracted into an equal volume of ethyl acetate and the pH is adjusted to less than 4 by the addition of an acid, the ethyl acetate phase is separated and then the solvent is removed by evaporation, then the sterol of interest is purified using chromatography.

### Example 14: 3-KCA Conversion Using Whole Cells of Pichia pastoris Strains Pichia pastoris Sand104—Pichia pastoris Sand121 Grown on YPD Medium

Conversion of 3-keto-5-beta-cholanic acid (3-KCA) acid to 3-keto-7-beta-hydroxy-5-beta-cholanic acid (3-KUDCA) by strains Pichia pastoris SAND104, Pichia pastoris SAND105, Pichia pastoris SAND106, Pichia pastoris SAND107, Pichia pastoris SAND108, Pichia pastoris SAND109, Pichia pastoris SAND110, Pichia pastoris SAND111, Pichia pastoris SAND112, Pichia pastoris SAND113, Pichia pastoris SAND114, Pichia pastoris SAND115, Pichia pastoris SAND116, Pichia pastoris SAND117, Pichia pastoris SAND118, Pichia pastoris SAND119, Pichia pastoris SAND120, and Pichia pastoris SAND121 is tested by induction of gene expression using standard methods. In one such method, YPD medium, containing 100 μg/ml nourseothricin and 100 μg/ml zeocin, is inoculated with a fresh single colony of the strain and incubated overnight at 30° C. shaking at 250 RPM. Fresh BMMY medium containing 2 mM aminolevulinic acid, 100 μl/ml nourseothricin and 100 μg/ml zeocin is inoculated with 1/10th volume overnight culture and incubated at 30° C. shaking at 250 RPM until an OD600 of 1.0 is reached. Methanol is added to a final concentration of 0.5% (v/v), 3-KCA is added to a final concentration of 1 mM and incubation is resumed at 30° C. shaking at 250 RPM for 2-3 days.

Products, including 3-KUDCA, are extracted from the broth using standard methods. In one method, the culture is extracted into an equal volume of ethyl acetate and the pH is adjusted to less than 4 by the addition of an acid, the ethyl acetate phase is separated and then the solvent is removed by evaporation, then the sterol of interest is purified using chromatography.

### Example 15: 3-Kca Conversion Using Whole Cells of Pichia pastoris Strains Pichia pastoris Sand104—Pichia pastoris Sand121 Grown on Bmg Medium

Conversion of 3-KCA to 3-KUDCA by strains Pichia pastoris SAND104, Pichia pastoris SAND105, Pichia pastoris SAND106, Pichia pastoris SAND107, Pichia pastoris SAND108, Pichia pastoris SAND109, Pichia pastoris SAND110, Pichia pastoris SAND111, Pichia pastoris SAND112, Pichia pastoris SAND113, Pichia pastoris SAND114, Pichia pastoris SAND115, Pichia pastoris SAND116, Pichia pastoris SAND117, Pichia pastoris SAND118, Pichia pastoris SAND119, Pichia pastoris SAND120, and Pichia pastoris SAND121 is tested by induction of gene expression using standard methods, such as that described in W. Lu, J. Feng, X. Chen, et al., 2019 Appl. Environ. Microbiol. 85, e01182-19. In this method, 25 ml BMG medium is inoculated with a fresh single colony of the strain and incubated at 30° C. shaking at 250 RPM to an OD600 of 10. Cells are harvested by centrifugation at 4000×g for 5 minutes and suspended in BMM medium containing 2 mM aminolevulenic acid to an OD600 of 1.0. The culture is incubated at shaking at 250 RPM with addition of methanol (1% v/v) every 24 hours for 5 days.

Cells are harvested by centrifugation at 4000×g for 5 minutes and resuspended in 30 ml mM potassium phosphate buffer, pH 7.5 containing 2 mM aminolevulenic acid and 1 mM 3-KCA. The cell suspension is incubated at 30° C. shaking at 200 RPM with addition of methanol (1% v/v) every 24 hours for 3 days.

Products, including 3-KUDCA, are extracted from the broth using standard methods. In one method, the culture is extracted into an equal volume of ethyl acetate and the pH is adjusted to less than 4 by the addition of an acid, the ethyl acetate phase is separated and then the solvent is removed by evaporation, then the sterol of interest is purified using chromatography.

### General Methods for Examples 16-21

### Analysis of Culture Extracts

Following solvent extraction of liquid cultures as described in the Examples, the samples were analyzed for production of UDCA and 3-KUDCA on an Agilent 1100 HPLC with a Waters XSelect CSH C18 column, (2.1 mm×50 mm×3.5 μm) fitted with a Waters VanGuard and an Acquity in line column filter and operated at 60° C. The mobile phase consisted of solvent A (0.005 M ammonium acetate, 0.012% formic acid) and solvent B (95% methanol, 5% water, 0.012% formic acid) with a flow rate of 1.0 mL/minute. A gradient was run from 50% solvent B to 100% solvent B over 9.5 minutes. Samples were analyzed by UV at 212 nm and by MS using a Waters ZQ single quadrupole MS running in electrospray negative ion mode with a mass range m/z of 150-500).

### Media

2TY medium contains 16 g/L bacto-tryptone, 10 g/L yeast extract and 5 g/L NaCl and is sterilized by autoclaving. 2TY agar additionally contains 15 g/L agar.

Synthetic Dextrose Minimal Medium contains 6.7 g/L yeast nitrogen base without amino acids, 20 g/L dextrose and 1.3 g/L amino acid dropout powder and is sterilized by autoclaving. Synthetic Dextrose Minimal Agar Medium contains 20 g/L agar.

Synthetic Galactose Minimal Medium contains 6.7 g/L yeast nitrogen base without amino acids, 20 g/L galactose and 1.3 g/L amino acid dropout powder and is sterilized by autoclaving. Synthetic Galactose Minimal Agar Medium contains 20 g/L agar.

Transformation of Pichia pastoris

Pichia pastoris (Komagataella phaffi NRRL Y-11430/ATCC 76273, hereafter referred to as Pichia pastoris SAND101) was grown overnight in 10 mL YPD at 30° C., shaking at 250 RPM. This culture was used to inoculate 500 mL YPD to an OD600 of 0.1, which was then incubated at shaking at 250 RPM to an OD600 of 1.3-1.5. Cells were harvested by centrifugation at 2000 xg at 4° C. for 10 minutes and resuspended in 100 mL YPD supplemented with 20 mL 1 M HEPES, pH 8.0 and 2.5 mL 1 M DTT. Cells were incubated at 30° C. without shaking for 15 minutes. Cold dH₂O was added to a final volume of 500 mL and cells were harvested by centrifugation at 2000 xg at 4° C. for 10 minutes. Cells were washed with 250 mL cold dH₂O and harvested by centrifugation at 2000×g at 4° C. for 10 minutes. Cells were washed with 20 mL cold 1 M sorbitol and harvested by centrifugation at 2000×g at 4° C. for 10 minutes. Cells were resuspended in 500 μl cold 1 M sorbitol. 100 ng DNA was added to 40 μl of the competent cells and transferred to a 2 mm gap electroporation cuvette, precooled on ice. Cells were electroporated on a BTRX ECM 630 decay wave electroporation system, using 1500 V, 200 Ω, 25 μF settings. 1 mL cold 1 M sorbitol was added immediately, and the mixture was transferred to a sterile Eppendorf tube. Cells were regenerated at 30° C., shaking at 250 RPM for at least 30 minutes. Cells were then spread onto YPD agar plates containing appropriate antibiotics, then incubated at 30° C. for 2 days or until colonies became visible.

Transformation of Saccharomyces cerevisiae

Saccharomyces cerevisiae YPH499 (Agilent) was grown overnight in 10 mL YPD at 30° C., shaking at 250 RPM. This culture was used to inoculate 500 mL YPD to an OD600 of 0.1, which was then incubated at 30° C., shaking at 250 RPM to an OD600 of 1.3-1.5. Cells were harvested by centrifugation at 2000×g at 4° C. for 10 minutes and resuspended in 100 mL YPD supplemented with 20 mL 1 M HEPES, pH 8.0 and 2.5 mL 1 M DTT. Cells were incubated at 30° C. without shaking for 15 minutes. Cold dH₂O was added to a final volume of 500 mL and cells were harvested by centrifugation at 2000×g at 4° C. for 10 minutes. Cells were washed with 250 mL cold dH₂O and harvested by centrifugation at 2000×g at 4° C. for 10 minutes. Cells were washed with 20 mL cold 1 M sorbitol and harvested by centrifugation at 2000×g at 4° C. for 10 minutes. Cells were resuspended in 500 μl cold 1 M sorbitol. 100 ng DNA was added to 40 μl of the competent cells and transferred to a 2 mm gap electroporation cuvette, precooled on ice. Cells were electroporated on a BTRX ECM 630 decay wave electroporation system, using 1500 V, 200 Ω, 25 μF settings. 1 mL cold 1 M sorbitol was added immediately, and the mixture was transferred to a sterile Eppendorf tube. Cells were regenerated at 30° C., shaking at 250 RPM for at least 30 minutes. Cells were then spread onto Synthetic Dextrose Minimal Agar Medium, lacking uracil, then incubated at 30° C. for 3 days or until colonies became visible.

### Example 16: Construction of a Pichia pastoris Strain Capable of Expressing Seq Id No. 2 and Seq Id No. 32

Plasmid pSAND101 was constructed as follows. Plasmid pPICHOLI-1 (MoBiTec GmbH, Germany) was cleaved with restriction enzymes Bsal and PciI. SEQ ID NO. 34 was ordered as synthetic DNA (Integrated DNA Technologies) and inserted into cleaved pPICHOLI-1 by infusion cloning (Takara Bio), followed by transformation of E. coli using standard methods. Transformants were plated onto 2TY agar containing 100 μg/mL nourseothricin. Correct assembly of pSAND101 was confirmed by restriction digest.

Plasmid pSAND102 was constructed as follows. Plasmid pSAND101 was cleaved with restriction enzymes EcoRI and SalI. SEQ ID NO. 35 was ordered as synthetic DNA (Twist Bioscience) and cleaved with restriction enzymes EcoRI and SalI. The digested synthetic DNA was inserted into cleaved pSAND101 by ligation following standard methods. E. coli transformants were plated onto 2TY agar containing 100 μg/mL nourseothricin. Correct assembly of pSAND102 was confirmed by restriction digest.

Plasmid pSAND112 was constructed as follows. Plasmid pPICHOLI-1 was cleaved with restriction enzymes EcoRI and SalI. SEQ ID NO. 36 was ordered as synthetic DNA (Twist Bioscience) and cleaved with restriction enzymes EcoRI and SalI. The digested synthetic DNA was inserted into cleaved pPICHOLI-1 by ligation following standard methods. E. coli transformants were plated onto 2TY agar containing 100 μg/mL zeocin. Correct assembly of pSAND112 was confirmed by restriction digest.

Plasmid pSAND102 was linearized by digestion with the restriction enzyme PmeI. Linearized pSAND102 was used to transform Pichia pastoris SAND101 by electroporation using standard methods. The resulting strain was labelled Pichia pastoris SAND102.

Plasmid pSAND112 was used to transform Pichia pastoris SAND102 by electroporation using standard methods. The resulting strain was labelled Pichia pastoris SAND121.

### Example 17: Bioconversion of LCA to UDCA by Pichia pastoris Sand121

Pichia pastoris SAND121 was used to inoculate 25 mL BMG medium, supplemented with 100 μg/mL zeocin in a 250-mL Erlenmeyer flask and incubated at 30° C., shaking at 250 RPM for 2 days, to be used as the seed culture.

Cells from the seed culture were harvested by centrifugation and used to inoculate 250 mL BMM containing 2 mM 5-aminolevulinic acid (5-ALA) in a 1-L Erlenmeyer flask to an OD595 of 1.0 and incubated at 20° C. for 5 days, to be used as the expression culture. The expression culture was shaken at 170 RPM for 1 day, then at 250 RPM for the remaining 4 days. Methanol was fed to the expression culture to a concentration of 1% v/v, daily.

Cells from 80 mL expression culture were harvested by centrifugation, suspended in 30 mL filter-sterilized potassium phosphate buffer at pH 7.5 and transferred to a 250-mL Erlenmeyer flask. Cells from 80 mL expression culture were harvested by centrifugation, suspended in 30 mL filter-sterilized potassium phosphate buffer at pH 9 and transferred to a 250-mL Erlenmeyer flask. To each flask was added 0.25 mL aqueous 5-ALA solution (200 mM) and 0.35 mL methanol containing 38.8 mg/mL LCA. Both flasks, to be used as the bioconversion cultures, were incubated at 30° C. with shaking at 250 RPM. Bioconversion cultures were fed 0.35 mL methanol daily, after which incubation continued, for 2 days. Bioconversion cultures were then fed 1.0 mL methanol, after which incubation continued for 3 days.

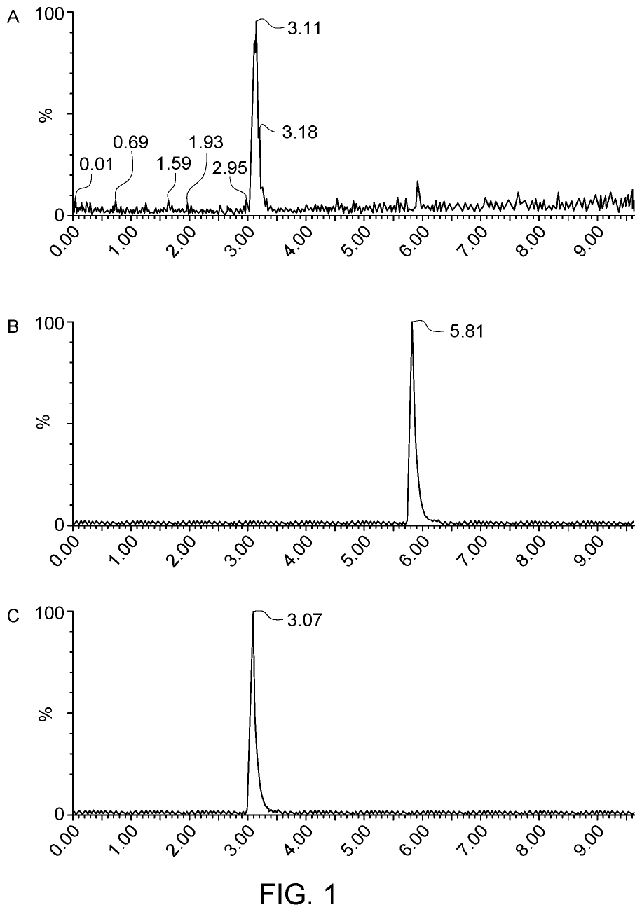

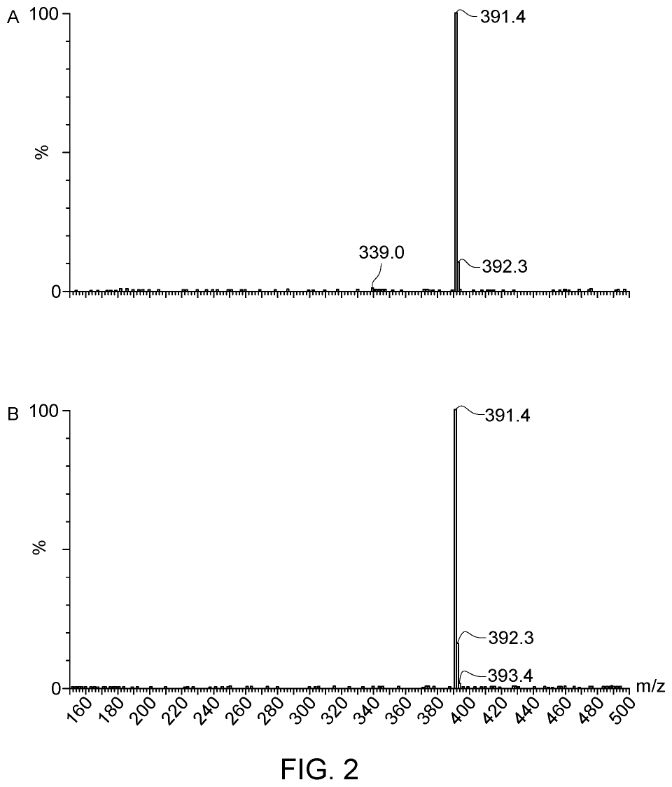

500 μL samples were withdrawn from the bioconversion culture and extracted with an equal volume of ethyl acetate containing 0.1% formic acid by shaking for 45 minutes. Phases were separated by centrifugation, and 20 μL of the solvent phase was transferred to a clean tube and evaporated. The pellet was dissolved in 20 μL of methanol, diluted 10-fold in a mixture of 50% mobile phase solution A and 50% mobile phase solution B, and analyzed by HPLC-MS (see General Methods). Peaks with an identical retention time and mass spectra profile as seen with the UDCA standard run alongside were seen (see FIG. 1 and FIG. 2).

Remaining bioconversion culture broths were transferred to 50-mL Falcon tubes and stored at −20° C. for later isolation of UDCA (see example 18).

### Example 18: Isolation of UDCA and Comparison with Authentic Standard

Bioconversion culture broths, stored at −20° C. as described in example 17, were thawed and centrifuged at 4500 RPM for 15 minutes. The resulting supernatant of 100 mL was decanted and extracted three times with an equal volume of ethyl acetate containing 0.1% formic acid, stirring for 45 minutes. The organic phases were pooled and evaporated under vacuum to yield a crude weighing 179 mg.

The crude was dissolved in 80 mL of ethyl acetate and dry loaded onto 1.5 g of silica-gel (Merck grade 9385, 200-400 mesh particle size) by removing the solvent in vacuo. The dried silica was poured on top of the pre-packed silica of a 25 g Biotage KP-Sil Snap cartridge (Biotage). The column was eluted with an ethyl acetate-hexane gradient of 10% ethyl acetate to 100% ethyl acetate over 10 column volumes. Fractions were collected and assayed by LCMS. Selected fractions were combined and the solvent evaporated on a rotary evaporator, yielding an extract weighing 11.3 mg.

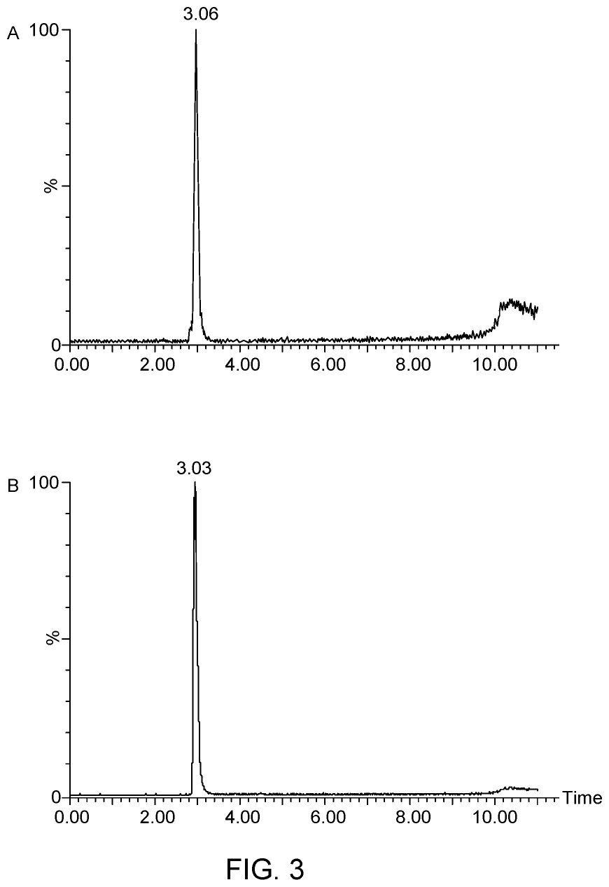

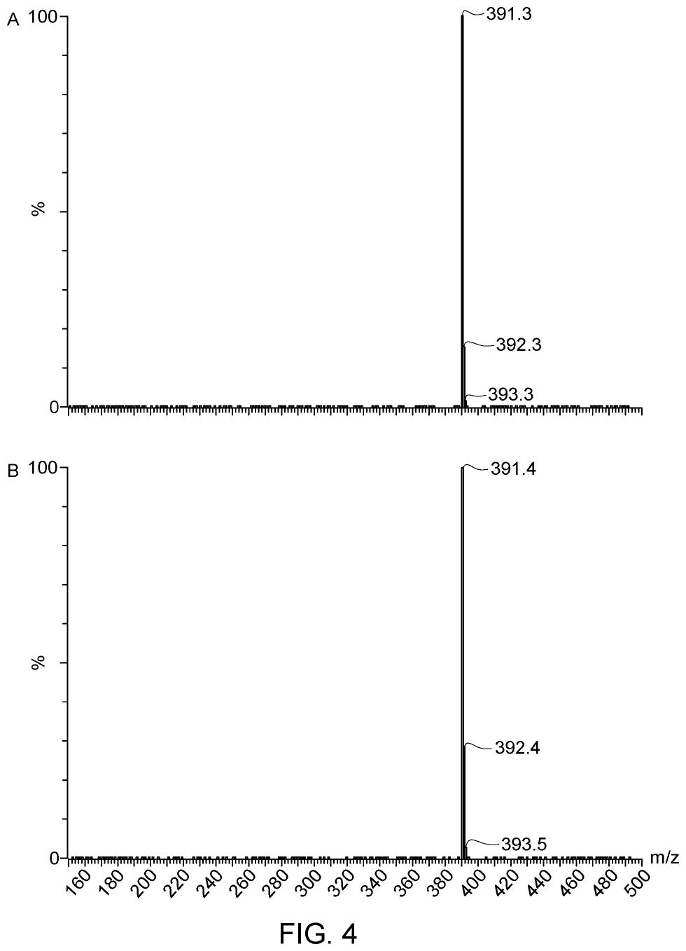

This extract was then dissolved in acetonitrile (0.3 mL) and DMSO (0.7 mL) and injected onto a 12 g Snap Ultra cartridge (Biotage) that had been pre-equilibrated with a mixture of 25% acetonitrile and 75% water. The column was eluted with an acetonitrile-water gradient of 25% acetonitrile to 80% acetonitrile over 10 column volumes. Fractions were collected and then assayed by LC-MS. Selected fractions were pooled, analyzed by LCMS (see FIG. 3 and FIG. 4) and then freeze-dried to yield a white powder weighing 3.8 mg.

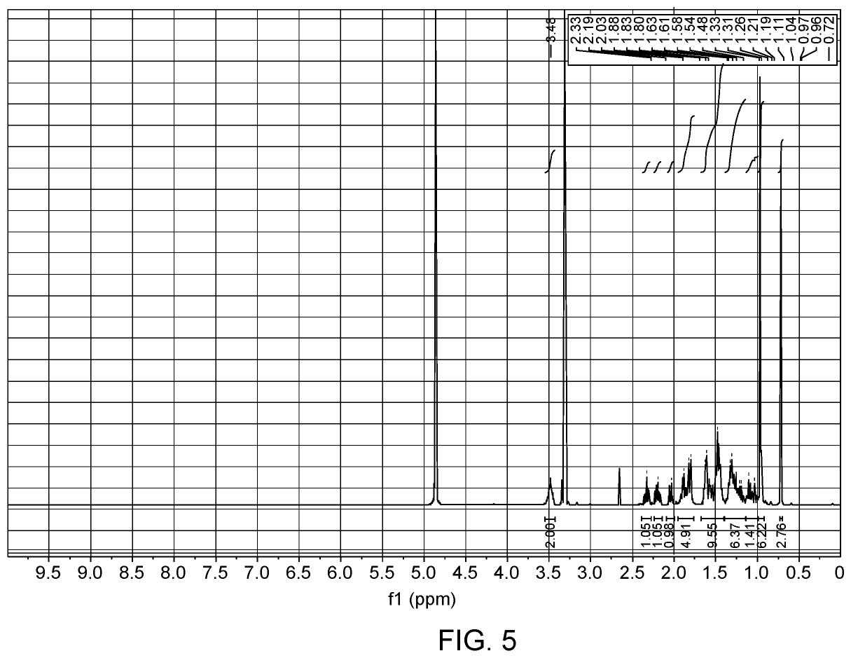

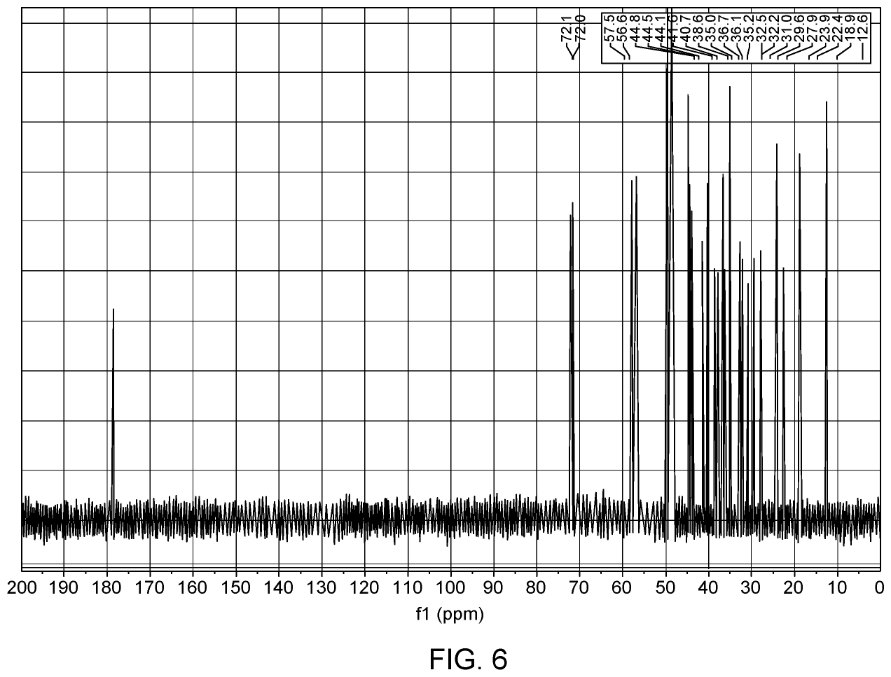

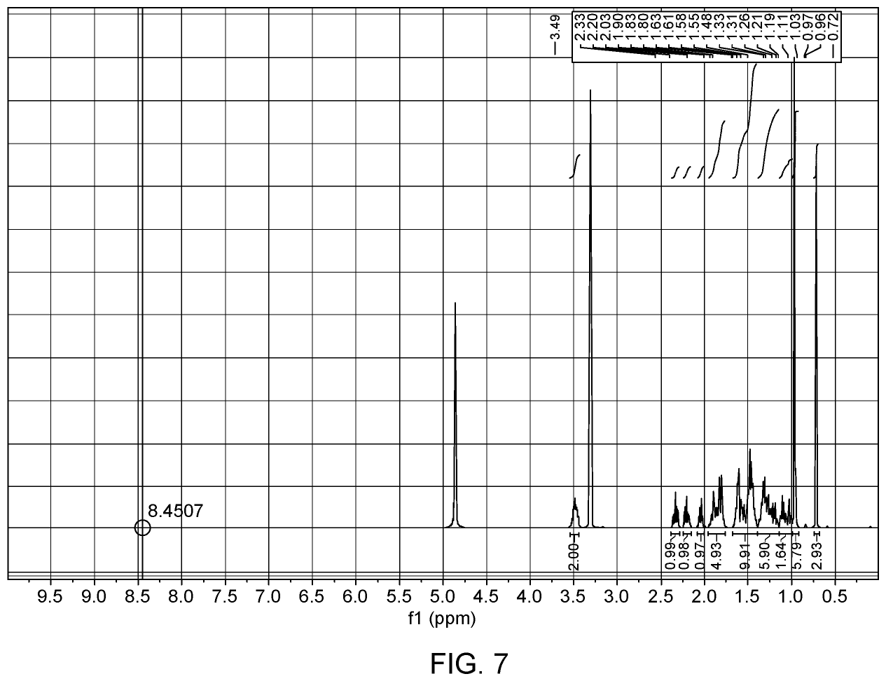

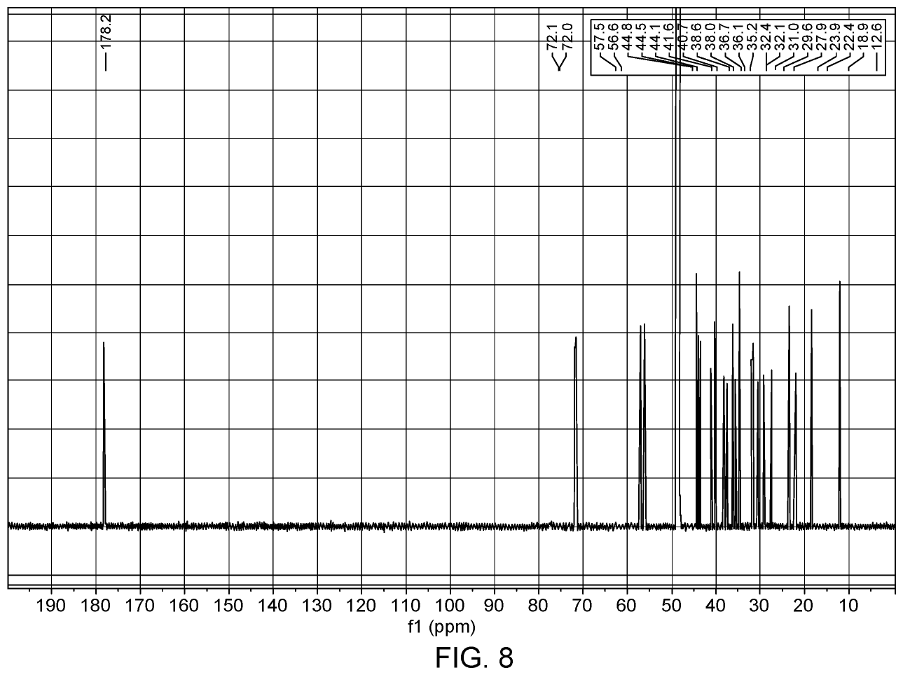

NMR spectroscopy in d4-methanol of this sample was undertaken and compared to a commercially obtained sample of UDCA (Sigma-Aldrich) which was run at the same time. NMR spectra were recorded on a Bruker 500 MHz DCH Cryoprobe Spectrometer at 298 K operating at 500.05 MHz and 125.75 MHz for 1H and 13C respectively. The UDCA commercially available standard NMR spectra was consistent with the sample NMR spectra (see FIG. 5, FIG. 6, FIG. 7 and FIG. 8).

### Example 19: Bioconversion of 3-Kca to 3-Kudca by Pichia pastoris Sand121

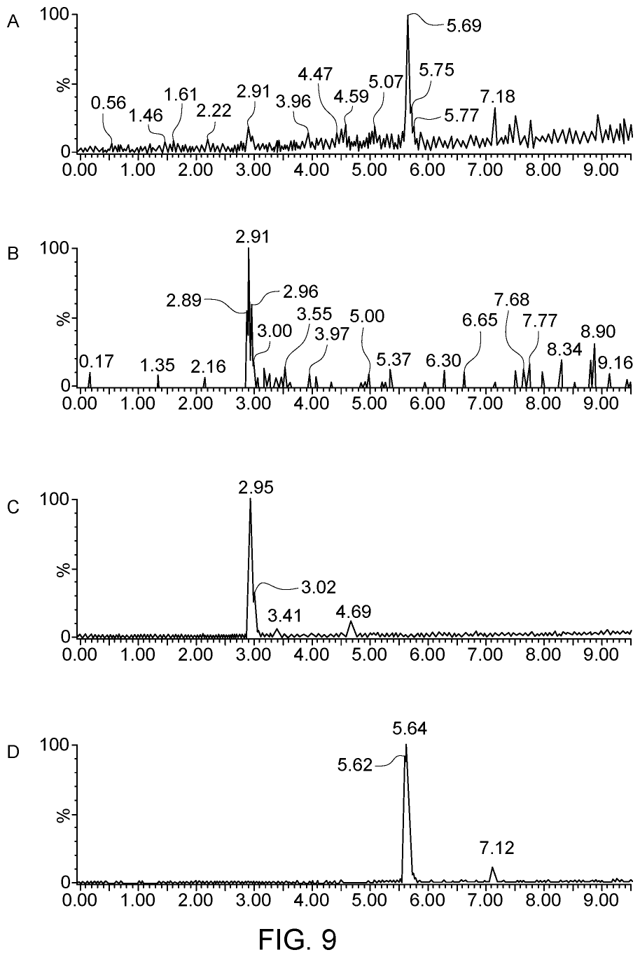

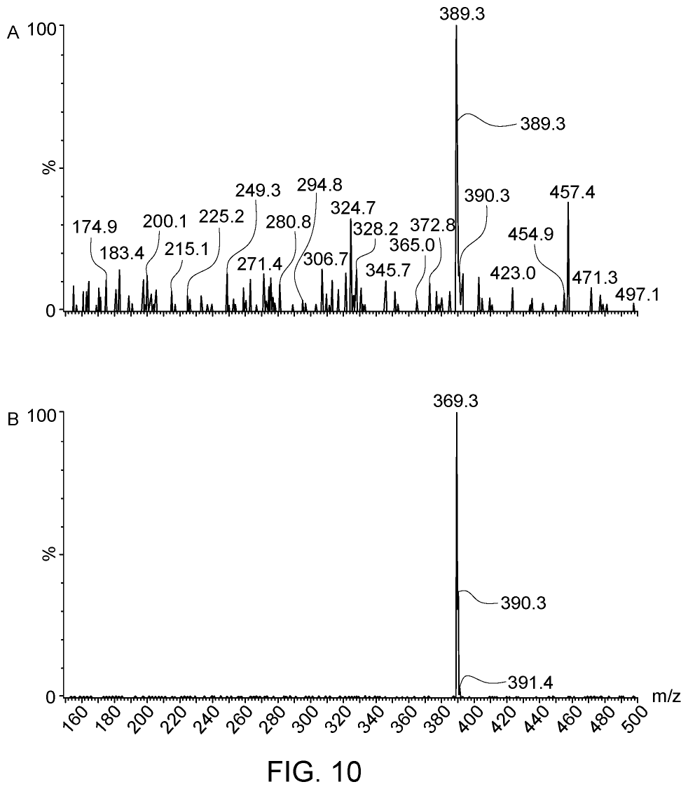

Pichia pastoris SAND121 was used to inoculate 25 mL BMG medium, supplemented with 100 μg/mL nourseothricin and 100 μg/mL zeocin in a 250-mL Erlenmeyer flask and incubated at shaking at 250 RPM for 3 days. 0.25 mL aqueous 5-ALA solution (200 mM) and 0.25 mL methanol containing 37.6 mg/mL 3-ketolithocholic acid (3-KCA) were added to the culture, then incubation was continued as before for 1 day. 0.25 mL methanol was added to the culture, then incubation was continued as before for 1 day. 800 μL broth was withdrawn from the culture and extracted with an equal volume of ethyl acetate containing 0.1% formic acid by shaking for 45 minutes. Phases were separated by centrifugation, and 400 μL of the solvent phase was transferred to a clean tube and evaporated. The pellet was dissolved in 400 μL of methanol by mixing for 10 minutes and centrifuged at 12000×g for 1 minute. 15 μL of the methanol solution was diluted 10-fold in a mixture of 50% mobile phase solution A and 50% mobile phase solution B, and analyzed by HPLC-MS (see General Methods). Peaks with an identical retention time and mass spectra profile as seen with the 3-KUDCA standard run alongside were seen (see FIG. 9 and FIG. 10).

### Example 20: Construction of Saccharomyces Cerevisiae Strains Capable of Expressing Seq Id No. 2 and Seq Id No. 32

Plasmid pSAND113, to express a gene encoding a P450 with sequence SEQ ID NO. 33, under control of the Gal1 promoter, and a gene encoding a P450 reductase with sequence SEQ ID NO. 3, under control of the Gall® promoter, was constructed as follows.

Plasmid pESC-URA (Agilent), was cleaved with restriction enzymes EcoRI and SpeI. An 837 bp fragment was amplified from plasmid pSAND102 using primers SEQ ID NO. 37 and SEQ ID NO. 38. This 837 bp fragment was inserted into EcoRI—SpeI digested pESC-URA using the SLiCE cloning method (Zhang et al., 2014), forming an intermediate plasmid. Insertion and identity of the insert were confirmed by restriction digest.

The intermediate plasmid was cleaved with restriction enzymes HindIII and SalI. A 1584 bp fragment was amplified from plasmid pSAND112 using primers SEQ ID NO. 39 and SEQ ID NO. 40. This 1584 bp fragment was inserted into the HindIII—SalI digested intermediate plasmid using the SLiCE cloning method (Zhang et al., 2014), forming plasmid pSAND113. Insertion and identity of the insert were confirmed by restriction digest.

Saccharomyces cerevisiae strain YPH499 (Agilent) was transformed with plasmid pSAND113 by electroporation, using standard methods, after which the cell suspension was plated onto Synthetic Dextrose Minimal Agar Medium, lacking uracil, and incubated at 30° C. until colonies were visible. The resulting strain was named Saccharomyces cerevisiae SAND122.

### Example 21: Bioconversion of Lca to Udca by Saccharomyces Cerevisiae Sand122

7 mL Synthetic Dextrose Minimal Medium, lacking uracil, in a 50-mL Falcon tube was inoculated with Saccharomyces cerevisiae SAND122 and incubated at 30° C. with shaking at 250 RPM for 24 hours, to be used as a seed culture.

1 mL of the seed culture was centrifuged briefly to harvest the cells. The supernatant was discarded, and the remaining cell pellet was suspended in 5 mL Synthetic Galactose Minimal Medium, lacking uracil, in a 50-mL Falcon tube capped with a foam bung. This culture was incubated at 30° C. with shaking at 250 RPM for 24 hours, to be used as the expression culture.

4 mL of the expression culture was briefly centrifuged to harvest the cells. The supernatant was discarded, and the remaining cell pellet was suspended in 5 mL bioconversion buffer (0.1 M potassium phosphate buffer at pH 10, 1% galactose and 650 mg/L LCA) in a 50-mL Falcon tube, capped with a foam bung. This suspension was incubated at 30° C. with shaking at 250 RPM for 72 hours, to be used as the bioconversion culture.

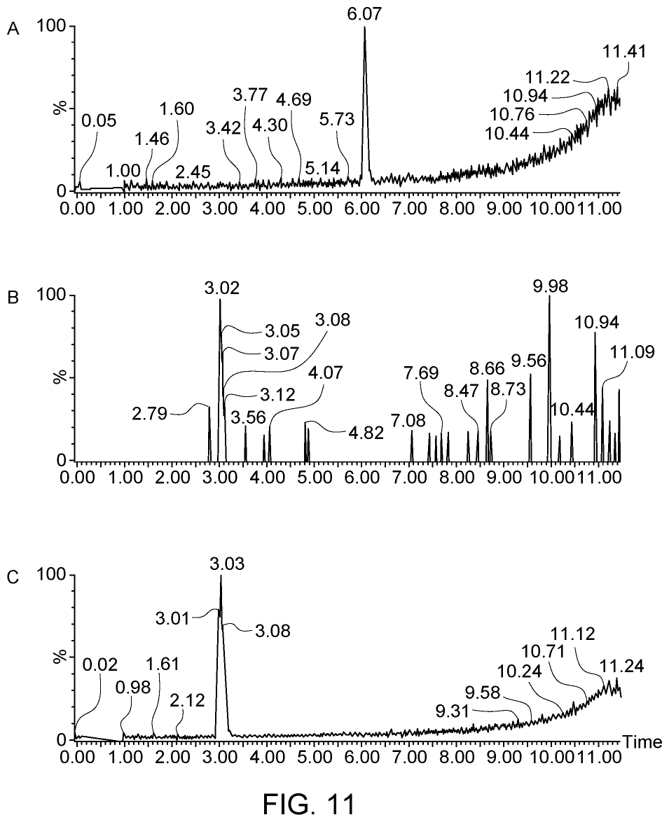

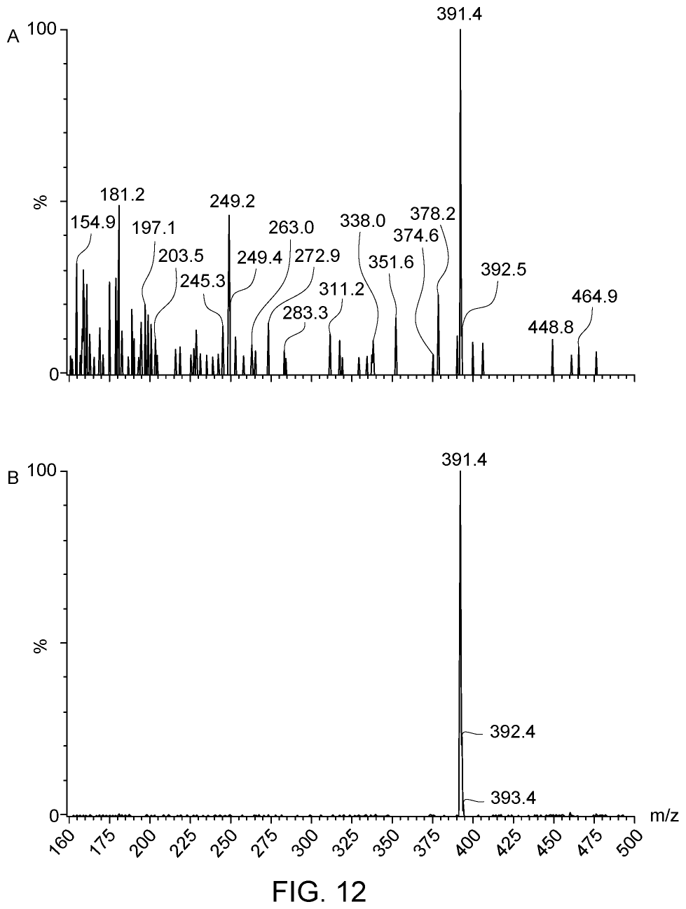

500 μL samples were withdrawn from the bioconversion culture and extracted with an equal volume of ethyl acetate containing 0.1% formic acid by shaking for 45 minutes. Phases were separated by centrifugation, and 20 μL of the solvent phase was transferred to a clean tube and evaporated. The pellet was dissolved in 20 μL of methanol, diluted 10-fold in a mixture of 50% mobile phase solution A and 50% mobile phase solution B, and analyzed by HPLC-MS (see General Methods). Peaks with an identical retention time and mass spectra profile as seen with the UDCA standard run alongside were observed (see FIG. 11 and FIG. 12).

### REFERENCES CITED

Zhang, Y., Werling, U., Ederlmann, W. (2014). Seamless Ligation Cloning Extract (SLiCE) Cloning Method. Methods in Molecular Biology 1116, 235-244.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of converting LCA or 3-KCA, or a carboxylic acid ester, carboxylic amide, or carboxylate salt thereof, to UDCA or 3-KUDCA, or a carboxylic acid ester, carboxylic amide, or carboxylate salt thereof, comprising contacting the LCA or 3-KCA, or carboxylic acid ester, carboxylic amide, or carboxylate salt thereof, with a 7β-hydroxylase system in the presence of a yeast, or an extract or lysate thereof, wherein the 7β-hydroxylase system is not native to the yeast.

2) The method of claim 1, wherein the yeast is selected from Saccharomyces and Pichia.

3) The method of claim 1, wherein the yeast is selected from Saccharomyces cerevisiae and Pichia pastoris.

4) (canceled)

5) The method of claim 4, wherein the 7β-hydroxylation system comprises a P450 oxidoreductase (“CPR”) enzyme and a P450 7-beta-hydroxylase (“CYP”) enzyme, the CYP enzyme is not native to the yeast, and the CPR enzyme can be native or not native to the yeast.

6) The method of claim 5, wherein the CYP enzyme is encoded by a CYP encoding nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; and SEQ ID NO. 32; or a nucleic acid sequence having at least 85% 90%, 95%, 98%, or 99%, identity with any of the foregoing nucleic acid sequences.

7) The method of claim 5, wherein the CPR enzyme is encoded by a CPR encoding nucleic acid sequence selected from SEQ ID NO. 2 and SEQ ID NO. 5, or a nucleic acid sequence having at least 85% 90%, 95%, 98%, or 99%, identity with any of the foregoing nucleic acid sequences.

8) The method of claim 5, wherein the CYP enzyme comprises a CYP amino acid sequence selected from SEQ ID NO. 9; SEQ ID NO. 12; SEQ ID NO. 15; SEQ ID NO. 18; SEQ ID NO. 21; SEQ ID NO. 24; SEQ ID NO. 27; SEQ ID NO. 30; or SEQ ID NO. 33; or an amino acid sequence having at least 85% 90%, 95%, 98%, or 99%, identity with any of the foregoing amino acid sequences.

9) The method of claim 5, wherein the CPR enzyme comprises a CPR amino acid sequence selected from SEQ ID NO. 3 and SEQ ID NO. 6, or an amino acid sequence having at least 85% 90%, 95%, 98%, or 99%, identity with any of the foregoing amino acid sequences.

10) The method of claim 1, wherein the 7β-hydroxylase system comprises a P450 7-beta-hydroxylase (“CYP”) enzyme native to F. graminearum or Gibberella zeae, preferably Gibberella zeae PH1 or Gibberella zeae VKM2600, most preferably Gibberella zeae VKM2600.

11) The method of claim 8, comprising contacting the LCA or carboxylic acid ester, carboxylic amide, or carboxylate salt thereof with the 7β-hydroxylase system to produce UDCA or a carboxylic acid ester, carboxylic amide, or carboxylate salt thereof.

12) The method of claim 8, comprising contacting the 3-KCA or carboxylic acid ester, carboxylic amide, or carboxylate salt thereof with the 7β-hydroxylase system to produce 3-KUDCA or a carboxylic acid ester, carboxylic amide, or carboxylate salt thereof.

13) The method of claim 12, further comprising reducing the 3-KUDCA or carboxylic acid ester, carboxylic amide, or carboxylate salt thereof to UDCA or a carboxylic acid ester, carboxylic amide, or carboxylate salt thereof.

14) (canceled)

15) (canceled)

16) (canceled)

17) (canceled)

18) (canceled)

19) A plasmid comprising a nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; or SEQ ID NO. 32; or a nucleic acid sequence having at least 85%, 90%, 95%, 98%, or 99%, identity with any of the foregoing sequences.

20) (canceled)

21) (canceled)

22) (canceled)

23) The plasmid of claim 19, under control of an AOX1 promoter and an AOX1 terminator sequence.

24) An organism transformed by a CYP encoding nucleic acid sequence selected from SEQ ID NO. 8; SEQ ID NO. 11; SEQ ID NO. 14; SEQ ID NO. 17; SEQ ID NO. 20; SEQ ID NO. 23; SEQ ID NO. 26; SEQ ID NO. 29; and SEQ ID NO. 32; or a nucleic acid sequence having at least 85%, 90%, 95%, 98%, or 99%, identity with any of the foregoing nucleic acid sequences.

25) (canceled)

26) (canceled)

27) (canceled)

28) The organism of claim 24, further transformed by a CPR encoding nucleic acid sequence comprising SEQ ID NO. 2 or SEQ ID NO. 5, or a nucleic acid sequence having at least 85% 90%, 95%, 98%, or 99%, identity with any of the foregoing nucleic acid sequences.

29) The organism of any of claim 24, wherein the organism is a yeast, preferably Saccharomyces or Pichia, and more preferably Saccharomyces cerevisiae or Pichia pastoris.

30-35) (canceled)

36) The method of claim 11, further comprising processing the UDCA into a finished dosage form.

37) The method of claim 13, further comprising processing the UDCA into a finished dosage form.


SEQUENCE LISTING (40 sequences)

SEQ ID NO: 1 - DNA, 4157 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggcccttcgaacgtccctatcacgacccgtaccgcttctggctacacttactgccagcgcaatcggagtatccatattgtctaaaatgatgttttcaacagcaagtgcagagagtccatctccgcaaaaaattttttccggtgcttttgcttccgtaaaactcccgctgcattcaagtgaatacgagtcccatgacacaaagaggcttcgtttcaaacttccgcaagagactgcagtaacgggtttaccgttagcttacttggttcacattccaccgtcccaccatcaaagggacttgactacgccggatgaacctggatacatggacctgttggtaaagaaataccccaaaggccagggctcgacatatctacactccctccagcccggtgatacgttatccttcacatctctacccctcaaaccagcttggaaaacaaacaattttcctcacatcactcttatagctggagggtgtgggatcacgccattattcaacttggctcaagggatacttagagatccggccgaaaaaactaggatgacctttatttttggtgcacgatcagacgaggacgtattactgaaaaaggagttagatggctttgcaaaagagttcccggaaagattcgaggtgaaatatacagcacttttggaagaggtcctagggggcgtgggtcgtgatactaaggtctttgtctgtgggccgaaggagatggaaaaggcacttgtaggaggccgtggcgtattaaaggaaataggcttcgaaaagtctcagatccatactttttgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaaggcgcgccgcaagccaagcctgcgaagaatgtagtcgagaattgagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgaccactttggatgatactgcttacagatacagaacttctgttccaggtgatgctgaagctattgaagctttggatggatctttcaccactgatactgttttcagagtcactgctactggtgatggattcactttgagagaagttcctgttgatcctcctttgaccaaagtttttcctgatgatgaatctgatgatgaatctgatgctggtgaagatggtgatccagattctagaacttttgttgcttatggtgatgatggtgatttggctggatttgttgttgtttcttattctggatggaacagaagattgactgttgaagatattgaagttgctccagaacatagaggtcatggtgttggaagagctttgatgggattggcaactgagtttgccagagaaagaggtgctggtcatctttggttggaagtcaccaatgtcaatgctccagctattcatgcttacagaagaatgggattcactctttgtggattggatactgctttgtatgatggaactgcttctgatggagaacaagctttgtacatgtccatgccatgtccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcggatctgtgagcaaacccgggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 2 - DNA, 795 bp, Artificial Sequence
  Synthetic DNA
atggcccttcgaacgtccctatcacgacccgtaccgcttctggctacacttactgccagcgcaatcggagtatccatattgtctaaaatgatgttttcaacagcaagtgcagagagtccatctccgcaaaaaattttttccggtgcttttgcttccgtaaaactcccgctgcattcaagtgaatacgagtcccatgacacaaagaggcttcgtttcaaacttccgcaagagactgcagtaacgggtttaccgttagcttacttggttcacattccaccgtcccaccatcaaagggacttgactacgccggatgaacctggatacatggacctgttggtaaagaaataccccaaaggccagggctcgacatatctacactccctccagcccggtgatacgttatccttcacatctctacccctcaaaccagcttggaaaacaaacaattttcctcacatcactcttatagctggagggtgtgggatcacgccattattcaacttggctcaagggatacttagagatccggccgaaaaaactaggatgacctttatttttggtgcacgatcagacgaggacgtattactgaaaaaggagttagatggctttgcaaaagagttcccggaaagattcgaggtgaaatatacagcacttttggaagaggtcctagggggcgtgggtcgtgatactaaggtctttgtctgtgggccgaaggagatggaaaaggcacttgtaggaggccgtggcgtattaaaggaaataggcttcgaaaagtctcagatccatactttttga

SEQ ID NO: 3 - protein, 264 aa, Fusarium graminearum
MALRTSLSRPVPLLATLTASAIGVSILSKMMFSTASAESPSPQKIFSGAFASVKLPLHSSEYESHDTKRLRFKLPQETAVTGLPLAYLVHIPPSHHQRDLTTPDEPGYMDLLVKKYPKGQGSTYLHSLQPGDTLSFTSLPLKPAWKTNNFPHITLIAGGCGITPLFNLAQGILRDPAEKTRMTFIFGARSDEDVLLKKELDGFAKEFPERFEVKYTALLEEVLGGVGRDTKVFVCGPKEMEKALVGGRGVLKEIGFEKSQIHTF

SEQ ID NO: 4 - DNA, 4400 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatgaaggaggctatcgttaagaaagatgcaagtgttgaggtagtggacagtccaataccgaaacctgggacgaatcctaaagattggaaaataccagccttttatggaacggagtctaattctggagatgacattgccgggttggttgaggcagtcggggaaaatgttgtaggtttccataaaggagacagggtggcagcttttcacgaaatgctgactccccatggagcctttgctgaatatgcaattgcacactattacactacgttccatattccagacagcatatcctacgaagaggctgccacgatacctttggctgcctatacttccgtatgcgccttgtttcaagagctacagttaccagatccttggagtcccctcgccaagttagacgagaaaagaccgttgctcgtatacggagcatcaacggctacggctgccttcgcaataaaactggccgctgccgcaaacgtacacccaatcatagccgtgggctctcaaagaagcgaatttgtaaaaccatttctagatgagtcaaagggcgacctattagtcgattacacgctgcacgatacagaagataaactggtggcagccatccaagacgcaattaaaaagtcaggtgcacccgacggtaggtgttgggtcgcatacgattcagtgtcagaggacagcaccgtccgtctggtgaccaaagcaatcgctggcccgccagatgcaaatggtcgaaaacctcgaatgacaaatttactcatgaaatccaacgtggaaggtgtggatccctctgtcgaaatagtacataccaaagtatctcaggtacacgaaaaaaacgaaaaagatcagatgttgggcctgacgtgggctgccgcatttagtaggggcctaagagagggatggcttactgctcacccctatatcgtgggaaagaacggactacagggactcagtgagggtctagtggccctgcgtgatggtaagacaaaagcaaataagttcctcactatactgtctgaaactcctggggctactgcttgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaaggcgcgccgcaagccaagcctgcgaagaatgtagtcgagaattgagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgaccactttggatgatactgcttacagatacagaacttctgttccaggtgatgctgaagctattgaagctttggatggatctttcaccactgatactgttttcagagtcactgctactggtgatggattcactttgagagaagttcctgttgatcctcctttgaccaaagtttttcctgatgatgaatctgatgatgaatctgatgctggtgaagatggtgatccagattctagaacttttgttgcttatggtgatgatggtgatttggctggatttgttgttgtttcttattctggatggaacagaagattgactgttgaagatattgaagttgctccagaacatagaggtcatggtgttggaagagctttgatgggattggcaactgagtttgccagagaaagaggtgctggtcatctttggttggaagtcaccaatgtcaatgctccagctattcatgcttacagaagaatgggattcactctttgtggattggatactgctttgtatgatggaactgcttctgatggagaacaagctttgtacatgtccatgccatgtccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcggatctgtgagcaaacccgggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 5 - DNA, 1038 bp, Artificial Sequence
  synthetic DNA
atgaaggaggctatcgttaagaaagatgcaagtgttgaggtagtggacagtccaataccgaaacctgggacgaatcctaaagattggaaaataccagccttttatggaacggagtctaattctggagatgacattgccgggttggttgaggcagtcggggaaaatgttgtaggtttccataaaggagacagggtggcagcttttcacgaaatgctgactccccatggagcctttgctgaatatgcaattgcacactattacactacgttccatattccagacagcatatcctacgaagaggctgccacgatacctttggctgcctatacttccgtatgcgccttgtttcaagagctacagttaccagatccttggagtcccctcgccaagttagacgagaaaagaccgttgctcgtatacggagcatcaacggctacggctgccttcgcaataaaactggccgctgccgcaaacgtacacccaatcatagccgtgggctctcaaagaagcgaatttgtaaaaccatttctagatgagtcaaagggcgacctattagtcgattacacgctgcacgatacagaagataaactggtggcagccatccaagacgcaattaaaaagtcaggtgcacccgacggtaggtgttgggtcgcatacgattcagtgtcagaggacagcaccgtccgtctggtgaccaaagcaatcgctggcccgccagatgcaaatggtcgaaaacctcgaatgacaaatttactcatgaaatccaacgtggaaggtgtggatccctctgtcgaaatagtacataccaaagtatctcaggtacacgaaaaaaacgaaaaagatcagatgttgggcctgacgtgggctgccgcatttagtaggggcctaagagagggatggcttactgctcacccctatatcgtgggaaagaacggactacagggactcagtgagggtctagtggccctgcgtgatggtaagacaaaagcaaataagttcctcactatactgtctgaaactcctggggctactgcttga

SEQ ID NO: 6 - protein, 345 aa, Fusarium graminearum
MKEAIVKKDASVEVVDSPIPKPGTNPKDWKIPAFYGTESNSGDDIAGLVEAVGENVVGFHKGDRVAAFHEMLTPHGAFAEYAIAHYYTTFHIPDSISYEEAATIPLAAYTSVCALFQELQLPDPWSPLAKLDEKRPLLVYGASTATAAFAIKLAAAANVHPIIAVGSQRSEFVKPFLDESKGDLLVDYTLHDTEDKLVAAIQDAIKKSGAPDGRCWVAYDSVSEDSTVRLVTKAIAGPPDANGRKPRMTNLLMKSNVEGVDPSVEIVHTKVSQVHEKNEKDQMLGLTWAAAFSRGLREGWLTAHPYIVGKNGLQGLSEGLVALRDGKTKANKFLTILSETPGATA

SEQ ID NO: 7 - DNA, 4418 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggactgtaaccccgactatgaaaatgccacctgggctttttatagatttgtccccagtaaagaagccaatattgtttttgtggtattgttcgccataaccacattgcttcatgtgctgcaactttggagaacacgaacgtggtacctaattccactcgtagtcgggggcgtaagtgccagtggcgaggtcataggatacataggccgagtattaaacacgaatgaagagcccggttgttggaccatgggcccatacataatgcagtccgtgttgatattaattgctcctgctctatttgcagcttctatttacatgatactgggccgtattatcattcttaccgaaggcgaacatcacagcctgatccctttaaagtggttaacgaagcttttcgtttttggggatgtcgcttcatttatgctacaatcaagtgggggtggcctgatggcaatacaggatttaaataagatgggagagaaaattatcgttggcggtttatttgtgcagcttttctttttcggttgttttattatagtctcagctgtgttccatatacgaatgcttagagctccgacgcctaacagttcgcaaactagggtacgatggcaaacatatttagcaactttgtacgtcactggtgtgcttatctgggtgcgatctttgttcagagtcattgagttcatagagggtaatgatggacacttgatgcgttcagaggtttgggttttcgttttcgatggcatgttaatgttattggtactcgtgtggatgaactggttccatcccggtgaaatcggccttctgataagaggagaagagtccataaccaacggattggaacttatgaaacttggtggcagtggtcgtaggtcccgagtggatacgatggagtcactgggcagcggcagacaccttgaggaaaataccgaaagataagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 8 - DNA, 939 bp, Artificial Sequence
  synthetic DNA
atggactgtaaccccgactatgaaaatgccacctgggctttttatagatttgtccccagtaaagaagccaatattgtttttgtggtattgttcgccataaccacattgcttcatgtgctgcaactttggagaacacgaacgtggtacctaattccactcgtagtcgggggcgtaagtgccagtggcgaggtcataggatacataggccgagtattaaacacgaatgaagagcccggttgttggaccatgggcccatacataatgcagtccgtgttgatattaattgctcctgctctatttgcagcttctatttacatgatactgggccgtattatcattcttaccgaaggcgaacatcacagcctgatccctttaaagtggttaacgaagcttttcgtttttggggatgtcgcttcatttatgctacaatcaagtgggggtggcctgatggcaatacaggatttaaataagatgggagagaaaattatcgttggcggtttatttgtgcagcttttctttttcggttgttttattatagtctcagctgtgttccatatacgaatgcttagagctccgacgcctaacagttcgcaaactagggtacgatggcaaacatatttagcaactttgtacgtcactggtgtgcttatctgggtgcgatctttgttcagagtcattgagttcatagagggtaatgatggacacttgatgcgttcagaggtttgggttttcgttttcgatggcatgttaatgttattggtactcgtgtggatgaactggttccatcccggtgaaatcggccttctgataagaggagaagagtccataaccaacggattggaacttatgaaacttggtggcagtggtcgtaggtcccgagtggatacgatggagtcactgggcagcggcagacaccttgaggaaaataccgaaagataa

SEQ ID NO: 9 - protein, 312 aa, Fusarium graminearum
MDCNPDYENATWAFYRFVPSKEANIVFVVLFAITTLLHVLQLWRTRTWYLIPLVVGGVSASGEVIGYIGRVLNTNEEPGCWTMGPYIMQSVLILIAPALFAASIYMILGRIIILTEGEHHSLIPLKWLTKLFVFGDVASFMLQSSGGGLMAIQDLNKMGEKIIVGGLFVQLFFFGCFIIVSAVFHIRMLRAPTPNSSQTRVRWQTYLATLYVTGVLIWVRSLFRVIEFIEGNDGHLMRSEVWVFVFDGMLMLLVLVWMNWFHPGEIGLLIRGEESITNGLELMKLGGSGRRSRVDTMESLGSGRHLEENTER

SEQ ID NO: 10 - DNA, 5072 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggaggccgtacacgccgacgtttcacaatacgaatatgccttagacgtagaagtgggtaaaaccgcacgactactgccactagaccttgactattgggtcagtggacagtacgcagctaggcttatgcacttgccgtatagtttacttgggaacgggggtaagcagtacccatacattaaccccaaaaagccattcgaacttagcaatcagcgtgttgtacaggattttatagagaatgctcgagacatactgactaaaggaaggtcgttatacaaagatacaccttataaagcacataccgacctgggggatgttctggttatacctccagaatttgccgatgctttaaagagcgaacgacaattagattttacagaggtagcaagagacgatacacacgggtacataccgggtttcgaaccgattggttctcctttcgatttagtacccttggtgaacaaatacctaactagggccctggccaaactgaccaagccgctgtgggccgaagcctctctgggagtcaaccatgtgttgggtacttcaacagaatggcatccgatcaatccaggagaggacataatgcgaatcgtctcccgtatgtcgtcaagaatatttatgggcgaggaactctgcaaggatgacgattggctcaaggtttctatagagtacactgtgcagttgttccaaacggcagacgagctaaggaactatccgagatggacacgtccgtacattcattggtttctccctagttgccaaggagtcaggcgtaaactacaggaggccagagatctgttgcaaccccatatagacaggcgtaacgccgtaaaaaaggaagcaatagctgaaggacgtccttcccccttcgacgatagcatcgaatggtttgaaaatgagtacgaaggaaagtctgacccggcaactgaacaaattaagctcagccttgtcgccatacacacaactacagacctgttgtctgaaactatgtttaatattgcactgcagcctgagttgctaggtcctcttcgtgaggaaatagttaccgtactatcgaccgagggtctaaagaaaacatctttctacaatttgaaacttatggactcggttataaaggagagccagaggcttcgaccggtcctattgggtgccttcagacgaatggcacttgctgatgtaacactgcctaatggcgacgtaattaagaaagggacaaaaattatctgtgatacaacccaccaatggaatccggagtactatccagacgcaagcaaattcaatgcctacaggtttcttcagatgaggcaaacaccagggcaagataaacgagcccacttagtctccacatcacacgatcaaatgggttttggccacggcctccacgcttgtccgggtagattctttgctgcaaacgaaattaaaatagccctatgccacatgttgttaaagtacgactggaagctacctgagggtgtcgttccgaaaagtaaggctctcggtatgtcactcctgggagacagagaggcaaaattgatggtcaagagaagggctgccgagatcgatatagacactattggtagtgacgaataggtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 11 - DNA, 1593 bp, Artificial Sequence
  synthetic DNA
atggaggccgtacacgccgacgtttcacaatacgaatatgccttagacgtagaagtgggtaaaaccgcacgactactgccactagaccttgactattgggtcagtggacagtacgcagctaggcttatgcacttgccgtatagtttacttgggaacgggggtaagcagtacccatacattaaccccaaaaagccattcgaacttagcaatcagcgtgttgtacaggattttatagagaatgctcgagacatactgactaaaggaaggtcgttatacaaagatacaccttataaagcacataccgacctgggggatgttctggttatacctccagaatttgccgatgctttaaagagcgaacgacaattagattttacagaggtagcaagagacgatacacacgggtacataccgggtttcgaaccgattggttctcctttcgatttagtacccttggtgaacaaatacctaactagggccctggccaaactgaccaagccgctgtgggccgaagcctctctgggagtcaaccatgtgttgggtacttcaacagaatggcatccgatcaatccaggagaggacataatgcgaatcgtctcccgtatgtcgtcaagaatatttatgggcgaggaactctgcaaggatgacgattggctcaaggtttctatagagtacactgtgcagttgttccaaacggcagacgagctaaggaactatccgagatggacacgtccgtacattcattggtttctccctagttgccaaggagtcaggcgtaaactacaggaggccagagatctgttgcaaccccatatagacaggcgtaacgccgtaaaaaaggaagcaatagctgaaggacgtccttcccccttcgacgatagcatcgaatggtttgaaaatgagtacgaaggaaagtctgacccggcaactgaacaaattaagctcagccttgtcgccatacacacaactacagacctgttgtctgaaactatgtttaatattgcactgcagcctgagttgctaggtcctcttcgtgaggaaatagttaccgtactatcgaccgagggtctaaagaaaacatctttctacaatttgaaacttatggactcggttataaaggagagccagaggcttcgaccggtcctattgggtgccttcagacgaatggcacttgctgatgtaacactgcctaatggcgacgtaattaagaaagggacaaaaattatctgtgatacaacccaccaatggaatccggagtactatccagacgcaagcaaattcaatgcctacaggtttcttcagatgaggcaaacaccagggcaagataaacgagcccacttagtctccacatcacacgatcaaatgggttttggccacggcctccacgcttgtccgggtagattctttgctgcaaacgaaattaaaatagccctatgccacatgttgttaaagtacgactggaagctacctgagggtgtcgttccgaaaagtaaggctctcggtatgtcactcctgggagacagagaggcaaaattgatggtcaagagaagggctgccgagatcgatatagacactattggtagtgacgaatag

SEQ ID NO: 12 - protein, 530 aa, Fusarium graminearum
MEAVHADVSQYEYALDVEVGKTARLLPLDLDYWVSGQYAARLMHLPYSLLGNGGKQYPYINPKKPFELSNQRVVQDFIENARDILTKGRSLYKDTPYKAHTDLGDVLVIPPEFADALKSERQLDFTEVARDDTHGYIPGFEPIGSPFDLVPLVNKYLTRALAKLTKPLWAEASLGVNHVLGTSTEWHPINPGEDIMRIVSRMSSRIFMGEELCKDDDWLKVSIEYTVQLFQTADELRNYPRWTRPYIHWFLPSCQGVRRKLQEARDLLQPHIDRRNAVKKEAIAEGRPSPFDDSIEWFENEYEGKSDPATEQIKLSLVAIHTTTDLLSETMFNIALQPELLGPLREEIVTVLSTEGLKKTSFYNLKLMDSVIKESQRLRPVLLGAFRRMALADVTLPNGDVIKKGTKIICDTTHQWNPEYYPDASKFNAYRFLQMRQTPGQDKRAHLVSTSHDQMGFGHGLHACPGRFFAANEIKIALCHMLLKYDWKLPEGVVPKSKALGMSLLGDREAKLMVKRRAAEIDIDTIGSDE

SEQ ID NO: 13 - DNA, 4802 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggcagctacgctaattgtgttcgggggtttgctgctcttggcctggcttgtcaacatcgcttatcgatcgttgtttcaccccttagctaaatttccgggccctaaactagccgcagtctctgacatttggtatgctattaagtggacatctggtagatatccttttataatggaagagactcatcgtaagtacggggatgtcgttagaatagcccccaatgaactatcattcgcaacagttcaagcctatcaagacatctacggacacgcactaaaaggaaagaaaaagtttgtaaaatccaactggtatgatacagctggtgatcaccctggaatagtttcagtgcgtgaccctaaagagcactctcgacaaagaaagtatctatcacacgccttctctgcaaagagcctgagagggcaagaagtgctggttcatgggtatgtcaacttgttcctggaccagttaagggaccttgcatttggggaatcgttcgatgcagttgctaacggaaaaactcacttttgggttagcatcattatagacgccacatacactagcatgctatctgctcttaggaagcgagtaccgctagtcaacttgtacctgccattcgtcgtgcctaaagatgctaaggccacataccaaaaacatcgtgcacttacccgtgaaaaaatgctaaagaggcttgatatgcctaattccgaggacagaggtgattttttcgccagtttgctaaggaagggtggaaacgaagtgcccgagccagagctactgcagcaatctaacaccctgatagtagcaggttccgaaactacagccacatgtttgaccggcatagtattctgtctattgtccaaccccagctgccttgaagccttatctaacgaagtaaggtctagatttcagtcggatagtgaaatcacgggcgacgctacagctgatatgaaatacctgtctgcagttatagaagaggggttgagaatcttcccgcctgccccatttggcctgcccagaatttctccaggcgccgtgattgacggtcactatgtgccacctggtgtgacggtgagtgtcgatcattggaccacgaaacatgaccgtcgatactggaaagacccttatagttttattcccgagcgatggatcgatgaagggtttggcgacacaaagcaggcttcacaaccattttctctaggacccagagcatgcttggggatcaaccttgcttacctagaaatgcgaattatcattgcaaaaatggtatattgcttcgattgggaactcccacgattaatggtcagattccatccccataattaggtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 14 - DNA, 1323 bp, Artificial Sequence
  synthetic DNA
atggcagctacgctaattgtgttcgggggtttgctgctcttggcctggcttgtcaacatcgcttatcgatcgttgtttcaccccttagctaaatttccgggccctaaactagccgcagtctctgacatttggtatgctattaagtggacatctggtagatatccttttataatggaagagactcatcgtaagtacggggatgtcgttagaatagcccccaatgaactatcattcgcaacagttcaagcctatcaagacatctacggacacgcactaaaaggaaagaaaaagtttgtaaaatccaactggtatgatacagctggtgatcaccctggaatagtttcagtgcgtgaccctaaagagcactctcgacaaagaaagtatctatcacacgccttctctgcaaagagcctgagagggcaagaagtgctggttcatgggtatgtcaacttgttcctggaccagttaagggaccttgcatttggggaatcgttcgatgcagttgctaacggaaaaactcacttttgggttagcatcattatagacgccacatacactagcatgctatctgctcttaggaagcgagtaccgctagtcaacttgtacctgccattcgtcgtgcctaaagatgctaaggccacataccaaaaacatcgtgcacttacccgtgaaaaaatgctaaagaggcttgatatgcctaattccgaggacagaggtgattttttcgccagtttgctaaggaagggtggaaacgaagtgcccgagccagagctactgcagcaatctaacaccctgatagtagcaggttccgaaactacagccacatgtttgaccggcatagtattctgtctattgtccaaccccagctgccttgaagccttatctaacgaagtaaggtctagatttcagtcggatagtgaaatcacgggcgacgctacagctgatatgaaatacctgtctgcagttatagaagaggggttgagaatcttcccgcctgccccatttggcctgcccagaatttctccaggcgccgtgattgacggtcactatgtgccacctggtgtgacggtgagtgtcgatcattggaccacgaaacatgaccgtcgatactggaaagacccttatagttttattcccgagcgatggatcgatgaagggtttggcgacacaaagcaggcttcacaaccattttctctaggacccagagcatgcttggggatcaaccttgcttacctagaaatgcgaattatcattgcaaaaatggtatattgcttcgattgggaactcccacgattaatggtcagattccatccccataattag

SEQ ID NO: 15 - protein, 440 aa, Fusarium graminearum
MAATLIVFGGLLLLAWLVNIAYRSLFHPLAKFPGPKLAAVSDIWYAIKWTSGRYPFIMEETHRKYGDVVRIAPNELSFATVQAYQDIYGHALKGKKKFVKSNWYDTAGDHPGIVSVRDPKEHSRQRKYLSHAFSAKSLRGQEVLVHGYVNLFLDQLRDLAFGESFDAVANGKTHFWVSIIIDATYTSMLSALRKRVPLVNLYLPFVVPKDAKATYQKHRALTREKMLKRLDMPNSEDRGDFFASLLRKGGNEVPEPELLQQSNTLIVAGSETTATCLTGIVFCLLSNPSCLEALSNEVRSRFQSDSEITGDATADMKYLSAVIEEGLRIFPPAPFGLPRISPGAVIDGHYVPPGVTVSVDHWTTKHDRRYWKDPYSFIPERWIDEGFGDTKQASQPFSLGPRACLGINLAYLEMRIIIAKMVYCFDWELPRLMVRFHPHN

SEQ ID NO: 16 - DNA, 5003 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatgttccaccttctgatatatccactatgggtcttggtggcattattcgccgtcattatcgcaaacctgctatatcaacagctgccaagacgtcctgatgaacccccattagtctttcactggttcccatttttcggtaatgcagtcgcctatggattggatccctgtggctttttcgagaaatgcagggagaagcacggggacgtattcacattcattttatttggtcgaaaaattgtagcctgcctgggcgtggacggaaacgatttcgttctgaactctaggctccaagacgccaacgctgaagaggtctacgggccactcaccattcctgtatttggcagcgacgttgtctatgactgccctaattcgaagctaatggaacaaaagaaattcgtcaaattcggtttaacgcagaaggctttggagagtcatgtgcaacttatcgagagggaggtgttggattacgtcgagacagacccctcattcagtgggagaacatcaacaatagatgttccgaaagccatggctgagatcacaatcttcactgctagtcgtagtttgcagggcgaggaagtcaggagaaagctgactgcagagttcgcagccctctaccatgatctcgacctgggctttaggccggttaactttctgttcccttggttgccgctgccccataacaggaagcgtgacgctgcccacatcaaaatgagggaggtctatatggacattataaatgacagacgaaaagggggaatacgtaccgaggacggtacggatatgattgccaatttaatgggatgcacatataagaatggccagccagttcctgataaggagattgcacacatgatgattacgctgctcatggcaggtcaacactcatccagctcggcttcttcatggattgtcctgcatttagcctcgagtcctgacattacggaagagttgtaccaagagcaactcgtcaatttatcagtcaacggggcccttcccccgcttcagtactctgacctagacaaattgccgttgttacagaatgttgtaaaggaaacgctccgagttcattctagtattcatagtattcttaggaaagttaagcgtccgatgcaagtccccaactcaccatatactattaccacggataaggtcatcatggcctcccccacggtgacagcaatgtcagaagagtacttcgagaatgctaaaacgtggaaccctcacagatgggacaacagggctaaagaggaagtggataccgaggatgtaatagactatggatacggagctgtcagtaaaggaacaaagtctccttatctaccgtttggggcagggagacatcgatgcatcggcgaaaagttcgcatacgtgaatttgggggtcatagttgctacgcttgtgagaaacttcaggttatcgacaatagacggccgacctggtgttcctgaaaccgactatacatccctattctcccgaccggctcagccggccttcattcgatgggaacgaaggaaaaagatttaggtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 17 - DNA, 1524 bp, Artificial Sequence
  synthetic DNA
atgttccaccttctgatatatccactatgggtcttggtggcattattcgccgtcattatcgcaaacctgctatatcaacagctgccaagacgtcctgatgaacccccattagtctttcactggttcccatttttcggtaatgcagtcgcctatggattggatccctgtggctttttcgagaaatgcagggagaagcacggggacgtattcacattcattttatttggtcgaaaaattgtagcctgcctgggcgtggacggaaacgatttcgttctgaactctaggctccaagacgccaacgctgaagaggtctacgggccactcaccattcctgtatttggcagcgacgttgtctatgactgccctaattcgaagctaatggaacaaaagaaattcgtcaaattcggtttaacgcagaaggctttggagagtcatgtgcaacttatcgagagggaggtgttggattacgtcgagacagacccctcattcagtgggagaacatcaacaatagatgttccgaaagccatggctgagatcacaatcttcactgctagtcgtagtttgcagggcgaggaagtcaggagaaagctgactgcagagttcgcagccctctaccatgatctcgacctgggctttaggccggttaactttctgttcccttggttgccgctgccccataacaggaagcgtgacgctgcccacatcaaaatgagggaggtctatatggacattataaatgacagacgaaaagggggaatacgtaccgaggacggtacggatatgattgccaatttaatgggatgcacatataagaatggccagccagttcctgataaggagattgcacacatgatgattacgctgctcatggcaggtcaacactcatccagctcggcttcttcatggattgtcctgcatttagcctcgagtcctgacattacggaagagttgtaccaagagcaactcgtcaatttatcagtcaacggggcccttcccccgcttcagtactctgacctagacaaattgccgttgttacagaatgttgtaaaggaaacgctccgagttcattctagtattcatagtattcttaggaaagttaagcgtccgatgcaagtccccaactcaccatatactattaccacggataaggtcatcatggcctcccccacggtgacagcaatgtcagaagagtacttcgagaatgctaaaacgtggaaccctcacagatgggacaacagggctaaagaggaagtggataccgaggatgtaatagactatggatacggagctgtcagtaaaggaacaaagtctccttatctaccgtttggggcagggagacatcgatgcatcggcgaaaagttcgcatacgtgaatttgggggtcatagttgctacgcttgtgagaaacttcaggttatcgacaatagacggccgacctggtgttcctgaaaccgactatacatccctattctcccgaccggctcagccggccttcattcgatgggaacgaaggaaaaagatttag

SEQ ID NO: 18 - protein, 507 aa, Fusarium graminearum
MFHLLIYPLWVLVALFAVIIANLLYQQLPRRPDEPPLVFHWFPFFGNAVAYGLDPCGFFEKCREKHGDVFTFILFGRKIVACLGVDGNDFVLNSRLQDANAEEVYGPLTIPVFGSDVVYDCPNSKLMEQKKFVKFGLTQKALESHVQLIEREVLDYVETDPSFSGRTSTIDVPKAMAEITIFTASRSLQGEEVRRKLTAEFAALYHDLDLGFRPVNFLFPWLPLPHNRKRDAAHIKMREVYMDIINDRRKGGIRTEDGTDMIANLMGCTYKNGQPVPDKEIAHMMITLLMAGQHSSSSASSWIVLHLASSPDITEELYQEQLVNLSVNGALPPLQYSDLDKLPLLQNVVKETLRVHSSIHSILRKVKRPMQVPNSPYTITTDKVIMASPTVTAMSEEYFENAKTWNPHRWDNRAKEEVDTEDVIDYGYGAVSKGTKSPYLPFGAGRHRCIGEKFAYVNLGVIVATLVRNFRLSTIDGRPGVPETDYTSLFSRPAQPAFIRWERRKKI

SEQ ID NO: 19 - DNA, 5060 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatgggacttttgcaagaacttgccgggcaccccctagcacagcaattccaggaacttcctttgggtcaacaggttggaattggctttgccgtttttttggtcctctcggtagtccttaatgttctaaaccagcttttattcaggaatccaaatgaaccgccaatggtctttcattggttcccttttgtagggagcacaatcacgtacggtatggatccccctacatttttcagagaaaacagagctaaacatggcgacgtattcacctttattctcttgggaaagaaaactacggttgctgtcggcccggcaggaaatgacttcattttaaacggtaagcttaaggacgtatgtgctgaagagatctacacggttctcacaactccagtattcggcaaagatgtcgtttatgattgtccaaacgctaagttaatggaacaaaaaaagttcatgaaaattgctctcacgacagaggcatttagatcttatgtgcccataatcagttcagaagtcagagactactttaagagaagtccagacttcaagggaaagtccggtattgcagatataccaaaaaagatggctgagattacaatattcactgcttcccacgccctccaaggttcggctataagaagtaagtttgatgagagcttggcagctttgtatcacgatctagacatgggctttacaccgattaactttatgttacactgggcaccgctgccttggaacaggaagcgagatcacgctcaaagaacggtcgcaaaaatatatatggatacgattaaagagcgacgtgcaaaaggtaacaatgaatcagaacatgatatgatgaagcatctgatgaactcgacgtacaaaaatggaatacgagttcccgatcacgaggttgcacacatgatgatcgcactccttatggctggacagcatagttcttcaagtactagctcgtggataatgctgcgtttggctcagtatccccatatcatggaggaattatatcaggagcaggtaaagaatttaggggcagatctgcctccattgacatatgaggatctagccaaacttccgttgaatcaagctatcgtaaaagaaactttacgtttacatgctccaatccactctattatgagggctgtcaaatccccaatgcccgtacctggcaccaaatatgtgataccgacatcacacacacttctagctgcacccggtgtctcggctacggactctgcatttttcccaaatcctgatgaatgggaccctcacagatgggaggctgattcccctaactttcccaggatggcttcgaaaggagaggacgaggaaaaaatagattatgggtatggtttagtctcaaaaggctccgcttcgccgtatctgccctttggagctggtaggcaccgatgcattggggaacactttgctaatgctcaattacagacaatcgtagctgaagtcgtgagggaatttaaatttcgtaatgtcgatggaggtcacacgttaattgatactgattacgcctcattgttctcgcgacccttggaacccgctaacatccattgggaacgtagacaataggtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 20 - DNA, 1581 bp, Artificial Sequence
  synthetic DNA
atgggacttttgcaagaacttgccgggcaccccctagcacagcaattccaggaacttcctttgggtcaacaggttggaattggctttgccgtttttttggtcctctcggtagtccttaatgttctaaaccagcttttattcaggaatccaaatgaaccgccaatggtctttcattggttcccttttgtagggagcacaatcacgtacggtatggatccccctacatttttcagagaaaacagagctaaacatggcgacgtattcacctttattctcttgggaaagaaaactacggttgctgtcggcccggcaggaaatgacttcattttaaacggtaagcttaaggacgtatgtgctgaagagatctacacggttctcacaactccagtattcggcaaagatgtcgtttatgattgtccaaacgctaagttaatggaacaaaaaaagttcatgaaaattgctctcacgacagaggcatttagatcttatgtgcccataatcagttcagaagtcagagactactttaagagaagtccagacttcaagggaaagtccggtattgcagatataccaaaaaagatggctgagattacaatattcactgcttcccacgccctccaaggttcggctataagaagtaagtttgatgagagcttggcagctttgtatcacgatctagacatgggctttacaccgattaactttatgttacactgggcaccgctgccttggaacaggaagcgagatcacgctcaaagaacggtcgcaaaaatatatatggatacgattaaagagcgacgtgcaaaaggtaacaatgaatcagaacatgatatgatgaagcatctgatgaactcgacgtacaaaaatggaatacgagttcccgatcacgaggttgcacacatgatgatcgcactccttatggctggacagcatagttcttcaagtactagctcgtggataatgctgcgtttggctcagtatccccatatcatggaggaattatatcaggagcaggtaaagaatttaggggcagatctgcctccattgacatatgaggatctagccaaacttccgttgaatcaagctatcgtaaaagaaactttacgtttacatgctccaatccactctattatgagggctgtcaaatccccaatgcccgtacctggcaccaaatatgtgataccgacatcacacacacttctagctgcacccggtgtctcggctacggactctgcatttttcccaaatcctgatgaatgggaccctcacagatgggaggctgattcccctaactttcccaggatggcttcgaaaggagaggacgaggaaaaaatagattatgggtatggtttagtctcaaaaggctccgcttcgccgtatctgccctttggagctggtaggcaccgatgcattggggaacactttgctaatgctcaattacagacaatcgtagctgaagtcgtgagggaatttaaatttcgtaatgtcgatggaggtcacacgttaattgatactgattacgcctcattgttctcgcgacccttggaacccgctaacatccattgggaacgtagacaatag

SEQ ID NO: 21 - protein, 526 aa, Fusarium graminearum
MGLLQELAGHPLAQQFQELPLGQQVGIGFAVFLVLSVVLNVLNQLLFRNPNEPPMVFHWFPFVGSTITYGMDPPTFFRENRAKHGDVFTFILLGKKTTVAVGPAGNDFILNGKLKDVCAEEIYTVLTTPVFGKDVVYDCPNAKLMEQKKFMKIALTTEAFRSYVPIISSEVRDYFKRSPDFKGKSGIADIPKKMAEITIFTASHALQGSAIRSKFDESLAALYHDLDMGFTPINFMLHWAPLPWNRKRDHAQRTVAKIYMDTIKERRAKGNNESEHDMMKHLMNSTYKNGIRVPDHEVAHMMIALLMAGQHSSSSTSSWIMLRLAQYPHIMEELYQEQVKNLGADLPPLTYEDLAKLPLNQAIVKETLRLHAPIHSIMRAVKSPMPVPGTKYVIPTSHTLLAAPGVSATDSAFFPNPDEWDPHRWEADSPNFPRMASKGEDEEKIDYGYGLVSKGSASPYLPFGAGRHRCIGEHFANAQLQTIVAEVVREFKFRNVDGGHTLIDTDYASLFSRPLEPANIHWERRQ

SEQ ID NO: 22 - DNA, 4994 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatgggagtcaataacgcgactttgggcttggtatgctgtgttatcgtcgcggtggttgctttagcgacgcgaaaggggcctgactcaagagagcccccgtatgttaaggaaagggtcccctacttcagtcacatctacggacttttgaagcatggcttacgttattttgatgttgtcagtgctcagcaaccccaccccatatttacgatagatatgtcgggccagaagaactatatagtaacttctcctgaactggttcaagcggtgcaacgcaacacaacgtcgttgagcttctccccggcaatgattcccgcttttcgacgcatgatggggtttgatgaagctgggatcgagctgatttttcgggatgcacatacagaaaaaggcatgtacggggaaattcacagggtccagaaggcgtctttacttccgggaactgagtcgttggacgaactttgcaccattatacgaggtaagttgttaacaattgtgaatgacatgccctcctctcaaacaatcgatctgtacgcgtgggtccaggacctttacatgaggacaaataactctgcttgctttggcgcaaaggatccttttactttaaacccgtccctgatttcgaccttctggttgtgggaggcgaatattaaggtattgttactggggattccatggttcctatccccctcaaaatattcaactgctcagcgaactagaaacgatttagtgaacgcgttcacgcaatacttgggtaatgatgggcttgaaactgcttgtagctttatcaaagaactatctaatttggggattcgtagaggccttagtaccgaaaataacgcgagggcgctggtcggcagcatcctggcaatcgtggggaatacaattccgacaaccttttggcttctcattcagatcttctccaggccagacctgctcaaggagatacgttctgagcttgaggcaacgctggaagatccatctagtcgatcagaaatatcactcaactatactgtgatcagagaaaagtgtccagttcttatgtctacatatgaggaaattctcaggatgacgagcggtatcgcaacagtcaggtacacgaatgaggatacgttaatccaggaccgctggttgttaaagaaaggcgcacaagtgcaaatgcccactgccttcatacatgccgacccaaccacgtggggcgcagacgcggaggtctttgatcacactaggttcttgaaatctaaggttctgacaaaagagcaaaaagcgcgcagagccgctgccttccggccttttgggggtggcaacaccctgtgcccgggacggcacttcgcgtcttatgaggtgcttaccttcgccgggagcatcctgctcggttttgatatgacacccacaactgaagctttcaacctccccgagatggataggtctaagcttcctctgacctccctgaaaccagctggggatatcaaagtcaacctaacccgccgttccgggtgggagaaggtgcaattcaagtgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 23 - DNA, 1515 bp, Artificial Sequence
  synthetic DNA
atgggagtcaataacgcgactttgggcttggtatgctgtgttatcgtcgcggtggttgctttagcgacgcgaaaggggcctgactcaagagagcccccgtatgttaaggaaagggtcccctacttcagtcacatctacggacttttgaagcatggcttacgttattttgatgttgtcagtgctcagcaaccccaccccatatttacgatagatatgtcgggccagaagaactatatagtaacttctcctgaactggttcaagcggtgcaacgcaacacaacgtcgttgagcttctccccggcaatgattcccgcttttcgacgcatgatggggtttgatgaagctgggatcgagctgatttttcgggatgcacatacagaaaaaggcatgtacggggaaattcacagggtccagaaggcgtctttacttccgggaactgagtcgttggacgaactttgcaccattatacgaggtaagttgttaacaattgtgaatgacatgccctcctctcaaacaatcgatctgtacgcgtgggtccaggacctttacatgaggacaaataactctgcttgctttggcgcaaaggatccttttactttaaacccgtccctgatttcgaccttctggttgtgggaggcgaatattaaggtattgttactggggattccatggttcctatccccctcaaaatattcaactgctcagcgaactagaaacgatttagtgaacgcgttcacgcaatacttgggtaatgatgggcttgaaactgcttgtagctttatcaaagaactatctaatttggggattcgtagaggccttagtaccgaaaataacgcgagggcgctggtcggcagcatcctggcaatcgtggggaatacaattccgacaaccttttggcttctcattcagatcttctccaggccagacctgctcaaggagatacgttctgagcttgaggcaacgctggaagatccatctagtcgatcagaaatatcactcaactatactgtgatcagagaaaagtgtccagttcttatgtctacatatgaggaaattctcaggatgacgagcggtatcgcaacagtcaggtacacgaatgaggatacgttaatccaggaccgctggttgttaaagaaaggcgcacaagtgcaaatgcccactgccttcatacatgccgacccaaccacgtggggcgcagacgcggaggtctttgatcacactaggttcttgaaatctaaggttctgacaaaagagcaaaaagcgcgcagagccgctgccttccggccttttgggggtggcaacaccctgtgcccgggacggcacttcgcgtcttatgaggtgcttaccttcgccgggagcatcctgctcggttttgatatgacacccacaactgaagctttcaacctccccgagatggataggtctaagcttcctctgacctccctgaaaccagctggggatatcaaagtcaacctaacccgccgttccgggtgggagaaggtgcaattcaagtga

SEQ ID NO: 24 - protein, 504 aa, Fusarium graminearum
MGVNNATLGLVCCVIVAVVALATRKGPDSREPPYVKERVPYFSHIYGLLKHGLRYFDVVSAQQPHPIFTIDMSGQKNYIVTSPELVQAVQRNTTSLSFSPAMIPAFRRMMGFDEAGIELIFRDAHTEKGMYGEIHRVQKASLLPGTESLDELCTIIRGKLLTIVNDMPSSQTIDLYAWVQDLYMRTNNSACFGAKDPFTLNPSLISTFWLWEANIKVLLLGIPWFLSPSKYSTAQRTRNDLVNAFTQYLGNDGLETACSFIKELSNLGIRRGLSTENNARALVGSILAIVGNTIPTTFWLLIQIFSRPDLLKEIRSELEATLEDPSSRSEISLNYTVIREKCPVLMSTYEEILRMTSGIATVRYTNEDTLIQDRWLLKKGAQVQMPTAFIHADPTTWGADAEVFDHTRFLKSKVLTKEQKARRAAAFRPFGGGNTLCPGRHFASYEVLTFAGSILLGFDMTPTTEAFNLPEMDRSKLPLTSLKPAGDIKVNLTRRSGWEKVQFK

SEQ ID NO: 25 - DNA, 5051 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatgatattcgacaacttgtcgctcagcaacacgtgggttgtgttagtacttagcgcggtatttcttgtgctttcccgttttattgctccgacaatctcagagaacgagcctcccatcgtcaagccaagggcccccttcattggacacattatctccatgttgagggacggctccgatatctacgttaatttgtttaagcaaagaaaggaaccaatagttactttacccatgttaaatggaaaattatacgtgataaattctccagacctcatacaggccgcattgcgtaacaatgacatctctttcacaccgttcattcttgagtcgtcaaaagcaatgtgggggttatctgataatgcgatggcgagcatatctgaccttgccaacttgaaaggcggtatgcagattatccactcaaccctcggaggggagtcgcttcataaattgaacatatcgtctctgagtaggttcatgacttatttgaatcgcgttaaacccggcgaaaatattggtatagccgacacttatatttggctgagagacatgctcaccgacgctagcgcgaccgcggtctatggtcctaagaatccaataaccgtcgataaaatgcacctagtatggtactcgttactacaatccatttactctacttgttccaacagtggtcgagattacgataaacaagcgttacttgtcgcaataggcctcccttccttcgtgacaaaagccgcgataaatgctcgtctaaaggttaataacttgcttctgtcgtactataaaaatggtggcaaccatgaaaaaggggcgtctgaaatcatacaacagcgggcaacgtatctgcgaaagacagggttcacagatgacgatttgtcccacatggagttcatgatactatgggtaggagtgactaatactgcacccgttctattctggttgtttgtccacgttcttacgtctgctggctatacgagccgcgtgcgggctgagatagaggcgataacaataatcaccaagacgccagagggcagaaaagcaaccttcgatacccgtttactcgagaaatcctgcccattcctcaacgcgtgttaccaggaatgccttcgacattactctcactcgatcggtaatcgtcgagtcatgcaggatactgagatccaagattctcagggccgaaagtaccttctaaagaaaggcgttaacgttcaatggccgcctccggtcacacatttcaatacggaagtttggggccaggacgcggatgtatttcgtccagaaagatttatggacgtcactcctcaggacgaaaaaaagaggagaggcgccctgttatccttcggaggtggcaaacacctttgcccgggtagaaagttcgcgtacacagaattgctagggcttgtgggggttgtggctcttggcttcgaagttaagggtctggagctacccgaaagtaaatacgcaggaatcggcataggaggcaagatgcctgattgggagaatatggaaaaaggcttcggtctaagacgtcgagaggggtgggaggatgttacctgggtctttgatggagataattgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 26 - DNA, 1572 bp, Artificial Sequence
  synthetic DNA
atgatattcgacaacttgtcgctcagcaacacgtgggttgtgttagtacttagcgcggtatttcttgtgctttcccgttttattgctccgacaatctcagagaacgagcctcccatcgtcaagccaagggcccccttcattggacacattatctccatgttgagggacggctccgatatctacgttaatttgtttaagcaaagaaaggaaccaatagttactttacccatgttaaatggaaaattatacgtgataaattctccagacctcatacaggccgcattgcgtaacaatgacatctctttcacaccgttcattcttgagtcgtcaaaagcaatgtgggggttatctgataatgcgatggcgagcatatctgaccttgccaacttgaaaggcggtatgcagattatccactcaaccctcggaggggagtcgcttcataaattgaacatatcgtctctgagtaggttcatgacttatttgaatcgcgttaaacccggcgaaaatattggtatagccgacacttatatttggctgagagacatgctcaccgacgctagcgcgaccgcggtctatggtcctaagaatccaataaccgtcgataaaatgcacctagtatggtactcgttactacaatccatttactctacttgttccaacagtggtcgagattacgataaacaagcgttacttgtcgcaataggcctcccttccttcgtgacaaaagccgcgataaatgctcgtctaaaggttaataacttgcttctgtcgtactataaaaatggtggcaaccatgaaaaaggggcgtctgaaatcatacaacagcgggcaacgtatctgcgaaagacagggttcacagatgacgatttgtcccacatggagttcatgatactatgggtaggagtgactaatactgcacccgttctattctggttgtttgtccacgttcttacgtctgctggctatacgagccgcgtgcgggctgagatagaggcgataacaataatcaccaagacgccagagggcagaaaagcaaccttcgatacccgtttactcgagaaatcctgcccattcctcaacgcgtgttaccaggaatgccttcgacattactctcactcgatcggtaatcgtcgagtcatgcaggatactgagatccaagattctcagggccgaaagtaccttctaaagaaaggcgttaacgttcaatggccgcctccggtcacacatttcaatacggaagtttggggccaggacgcggatgtatttcgtccagaaagatttatggacgtcactcctcaggacgaaaaaaagaggagaggcgccctgttatccttcggaggtggcaaacacctttgcccgggtagaaagttcgcgtacacagaattgctagggcttgtgggggttgtggctcttggcttcgaagttaagggtctggagctacccgaaagtaaatacgcaggaatcggcataggaggcaagatgcctgattgggagaatatggaaaaaggcttcggtctaagacgtcgagaggggtgggaggatgttacctgggtctttgatggagataattga

SEQ ID NO: 27 - protein, 523 aa, Fusarium graminearum
MIFDNLSLSNTWVVLVLSAVFLVLSRFIAPTISENEPPIVKPRAPFIGHIISMLRDGSDIYVNLFKQRKEPIVTLPMLNGKLYVINSPDLIQAALRNNDISFTPFILESSKAMWGLSDNAMASISDLANLKGGMQIIHSTLGGESLHKLNISSLSRFMTYLNRVKPGENIGIADTYIWLRDMLTDASATAVYGPKNPITVDKMHLVWYSLLQSIYSTCSNSGRDYDKQALLVAIGLPSFVTKAAINARLKVNNLLLSYYKNGGNHEKGASEIIQQRATYLRKTGFTDDDLSHMEFMILWVGVTNTAPVLFWLFVHVLTSAGYTSRVRAEIEAITIITKTPEGRKATFDTRLLEKSCPFLNACYQECLRHYSHSIGNRRVMQDTEIQDSQGRKYLLKKGVNVQWPPPVTHFNTEVWGQDADVFRPERFMDVTPQDEKKRRGALLSFGGGKHLCPGRKFAYTELLGLVGVVALGFEVKGLELPESKYAGIGIGGKMPDWENMEKGFGLRRREGWEDVTWVFDGDN

SEQ ID NO: 28 - DNA, 5048 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggagtccatgataattactcctgagatgaactcaactttaaagatcgcggatgtccaagcccacgacttacctttgcaacacaactttctgtcatacttgtttggattgctaatcgccacatatatagtatggcagtatttcctgcgaactggagtcacggagtcagcttgctccgagcctccaatgctaccctattggatccccgtggtaggtcataccttcagtttcttgactaatactcataatacgataatgtcgggccggagtcacttcaaatctataacacatcccttctctctgttgattggaggtagaaggacttacgtagtccttgacccgcactatattggaaaggtgtacaagaaaacgaaagatttggttcatgagccgtttatagatcacttaatgatgtgcatcgggacaactcaaaaaacgagggacataatgtggaacacaatgatcggggactccagtctaaccgattcggctctcgattggcttagggaggaagtctcccaatcgccttctagccaaccatttttcgacagattcatgatggaattggatcatggcctccagcaaggcgacccgcttactacggggcgacttcgggaacataacatgcttaagtttgttgaaacaattataatcaccgtatcaactaatagcttctttgggaaggtgcttctaaaacaatctccagaaattcttgactcgtttccaatttttgaccgacacgtctggaagatggtattccgcgcaccaaaatttactttcatgacggcacacaacgcgaagggttctgtcatcgacggtcttactaaatattttgatttaccacaaagtgagagacaggacgccgcttcttttatccttaaaagtgaggacgcaatgcgtgagaatggaatctgctcacgggagattgcggccctgctctttaaattcttttggggcataaatggcatgcccgcgacactggccttctggtttcttgccaggactgtctacacaccacacctttgggaggatatacgtgcagaggtcgcaccggcctttaggaatggtattcattcacccccagacatagggtatttgaaaaagtgcccaaaattaaacgccaccttccacgaaacgttacgcatccacggtgggacggctggatttaggcaagtcgcgagtgataccgtcataggtggatttaccttcaaggccgggtccgacgttataatgccgtaccggcaaatgcacctagatgaggggatctgggggcaggacgctaagacttttgatattgatcgctttattcataacccgaaactagctaccgcaaagacatttaagccttttggaggcggtgtaacattgtgtccaggacgcttccatgcgcaccgaactgctctgagctttattgcgattgttataacccgatacgacatccacgttgtgggcggttgcgaatcgcgacccttcccacatatgaatacacgcggaccagaggttggtgttatattcccagtcttggagcaggtgccacaaattatagtaaaaaatgttgacattgaatgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 29 - DNA, 1569 bp, Artificial Sequence
  synthetic DNA
atggagtccatgataattactcctgagatgaactcaactttaaagatcgcggatgtccaagcccacgacttacctttgcaacacaactttctgtcatacttgtttggattgctaatcgccacatatatagtatggcagtatttcctgcgaactggagtcacggagtcagcttgctccgagcctccaatgctaccctattggatccccgtggtaggtcataccttcagtttcttgactaatactcataatacgataatgtcgggccggagtcacttcaaatctataacacatcccttctctctgttgattggaggtagaaggacttacgtagtccttgacccgcactatattggaaaggtgtacaagaaaacgaaagatttggttcatgagccgtttatagatcacttaatgatgtgcatcgggacaactcaaaaaacgagggacataatgtggaacacaatgatcggggactccagtctaaccgattcggctctcgattggcttagggaggaagtctcccaatcgccttctagccaaccatttttcgacagattcatgatggaattggatcatggcctccagcaaggcgacccgcttactacggggcgacttcgggaacataacatgcttaagtttgttgaaacaattataatcaccgtatcaactaatagcttctttgggaaggtgcttctaaaacaatctccagaaattcttgactcgtttccaatttttgaccgacacgtctggaagatggtattccgcgcaccaaaatttactttcatgacggcacacaacgcgaagggttctgtcatcgacggtcttactaaatattttgatttaccacaaagtgagagacaggacgccgcttcttttatccttaaaagtgaggacgcaatgcgtgagaatggaatctgctcacgggagattgcggccctgctctttaaattcttttggggcataaatggcatgcccgcgacactggccttctggtttcttgccaggactgtctacacaccacacctttgggaggatatacgtgcagaggtcgcaccggcctttaggaatggtattcattcacccccagacatagggtatttgaaaaagtgcccaaaattaaacgccaccttccacgaaacgttacgcatccacggtgggacggctggatttaggcaagtcgcgagtgataccgtcataggtggatttaccttcaaggccgggtccgacgttataatgccgtaccggcaaatgcacctagatgaggggatctgggggcaggacgctaagacttttgatattgatcgctttattcataacccgaaactagctaccgcaaagacatttaagccttttggaggcggtgtaacattgtgtccaggacgcttccatgcgcaccgaactgctctgagctttattgcgattgttataacccgatacgacatccacgttgtgggcggttgcgaatcgcgacccttcccacatatgaatacacgcggaccagaggttggtgttatattcccagtcttggagcaggtgccacaaattatagtaaaaaatgttgacattgaatga

SEQ ID NO: 30 - protein, 522 aa, Fusarium graminearum
MESMIITPEMNSTLKIADVQAHDLPLQHNFLSYLFGLLIATYIVWQYFLRTGVTESACSEPPMLPYWIPVVGHTFSFLTNTHNTIMSGRSHFKSITHPFSLLIGGRRTYVVLDPHYIGKVYKKTKDLVHEPFIDHLMMCIGTTQKTRDIMWNTMIGDSSLTDSALDWLREEVSQSPSSQPFFDRFMMELDHGLQQGDPLTTGRLREHNMLKFVETIIITVSTNSFFGKVLLKQSPEILDSFPIFDRHVWKMVFRAPKFTFMTAHNAKGSVIDGLTKYFDLPQSERQDAASFILKSEDAMRENGICSREIAALLFKFFWGINGMPATLAFWFLARTVYTPHLWEDIRAEVAPAFRNGIHSPPDIGYLKKCPKLNATFHETLRIHGGTAGFRQVASDTVIGGFTFKAGSDVIMPYRQMHLDEGIWGQDAKTFDIDRFIHNPKLATAKTFKPFGGGVTLCPGRFHAHRTALSFIAIVITRYDIHVVGGCESRPFPHMNTRGPEVGVIFPVLEQVPQIIVKNVDIE

SEQ ID NO: 31 - DNA, 5021 bp, Artificial Sequence
  plasmid
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatcctacgtattaatacgactcactatatttgctttgtgagcggataacaattataatagattcaattgtgagcggataacaatttcacacagaattcatggccacggatcttgacctcgtgctgggaaaaagtcagtacgcattattttgtggcataactttatttagctttttcatactaaagtattcccttctcggaaacgggggcaagcaatacccttatatcaaccccaagaaaccctttgagctgtcgaaccagcgagtagtccaggatttcatcgagaacgcacgagacattcttactaagggtcgctcactttacaaggatacgccctacaaggcgcataccgatttaggggacgtcctcgtaatcccgcccgagtttgccgacgctctcaagtccgaaagacagcttgactttaccgaggtcgcgagagacgatactcacggttacattcctggattcgagcccataggttccccgttcgatctggtgccgctcgtcaacaagtatcttacaagggcgttggcaaaactaacaaagccactgtgggccgaagcctcgttaggtgtaaaccatgttctgggcacgtctacggagtggcatcccattaacccaggcgaagatatcatgaggatagtctccagaatgtcatccagaatattcatgggtgaggaactttgtaaagatgacgattggctgaaagtgtcgattgagtacactgtgcagctgtttcaaaccgcagacgaattacgtaactatccacgttggacgcggccctatattcactggttcttgccttcctgtcagggggttcgtcgcaagttgcaggaggcgcgtgatttattgcaaccccatattgataggagaaatgcagtgaagaaagaagcgatcgctgaaggtagaccctcaccattcgacgattcaatagagtggtttgaaaatgagtacgagggcaaatctgatcccgccactgaacaaattaaactatcactggtggcgattcacacaaccacggacctcctgtctgaaaccatgttcaatatagctttgcagccagaactccttggtcccctacgtgaagagatagttacggttctttccacggaaggtctaaaaaagacgtcgttttacaatttgaagttgatggattcggtcataaaggagtcacagcgacttcgacccgttcttctcggtgcgttccgaagaatggcactcgctgacgtaaccttgcccaatggcgacgtaataaagaaagggaccaagatcatttgcgacactacacatcagtggaacccagaatactatcccgatgccagcaagttcaatgcatatcggtttctccaaatgagacagacgcccggtcaggacaaaagagcacaccttgtcagcacaagccacgatcaaatggggttcggacacggcttgcacgcgtgcccaggccggtttttcgcagccaatgagataaagatagcgctgtgtcacatgctattgaagtatgactggaagcttccagaaggtgttgtacctaagtctaaggccctcggcatgtccttactgggggaccgggaagccaaactgatggtcaagaggagagcagccgaaatcgatatagacactattgggagcgatgaatgagtcgacctgcaagatctgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatccaattaatatttacttattttggtcaaccccaaataggttgatttcatacttggttcattcaaaaataagtagtcttttgagatctttcaatattataataaatatactataacagccgacttgtttcattttcgcgaatgttcccccagcttatcggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 32 - DNA, 1542 bp, Artificial Sequence
  synthetic DNA
atggccacggatcttgacctcgtgctgggaaaaagtcagtacgcattattttgtggcataactttatttagctttttcatactaaagtattcccttctcggaaacgggggcaagcaatacccttatatcaaccccaagaaaccctttgagctgtcgaaccagcgagtagtccaggatttcatcgagaacgcacgagacattcttactaagggtcgctcactttacaaggatacgccctacaaggcgcataccgatttaggggacgtcctcgtaatcccgcccgagtttgccgacgctctcaagtccgaaagacagcttgactttaccgaggtcgcgagagacgatactcacggttacattcctggattcgagcccataggttccccgttcgatctggtgccgctcgtcaacaagtatcttacaagggcgttggcaaaactaacaaagccactgtgggccgaagcctcgttaggtgtaaaccatgttctgggcacgtctacggagtggcatcccattaacccaggcgaagatatcatgaggatagtctccagaatgtcatccagaatattcatgggtgaggaactttgtaaagatgacgattggctgaaagtgtcgattgagtacactgtgcagctgtttcaaaccgcagacgaattacgtaactatccacgttggacgcggccctatattcactggttcttgccttcctgtcagggggttcgtcgcaagttgcaggaggcgcgtgatttattgcaaccccatattgataggagaaatgcagtgaagaaagaagcgatcgctgaaggtagaccctcaccattcgacgattcaatagagtggtttgaaaatgagtacgagggcaaatctgatcccgccactgaacaaattaaactatcactggtggcgattcacacaaccacggacctcctgtctgaaaccatgttcaatatagctttgcagccagaactccttggtcccctacgtgaagagatagttacggttctttccacggaaggtctaaaaaagacgtcgttttacaatttgaagttgatggattcggtcataaaggagtcacagcgacttcgacccgttcttctcggtgcgttccgaagaatggcactcgctgacgtaaccttgcccaatggcgacgtaataaagaaagggaccaagatcatttgcgacactacacatcagtggaacccagaatactatcccgatgccagcaagttcaatgcatatcggtttctccaaatgagacagacgcccggtcaggacaaaagagcacaccttgtcagcacaagccacgatcaaatggggttcggacacggcttgcacgcgtgcccaggccggtttttcgcagccaatgagataaagatagcgctgtgtcacatgctattgaagtatgactggaagcttccagaaggtgttgtacctaagtctaaggccctcggcatgtccttactgggggaccgggaagccaaactgatggtcaagaggagagcagccgaaatcgatatagacactattgggagcgatgaatga

SEQ ID NO: 33 - protein, 513 aa, Fusarium graminearum
MATDLDLVLGKSQYALFCGITLFSFFILKYSLLGNGGKQYPYINPKKPFELSNQRVVQDFIENARDILTKGRSLYKDTPYKAHTDLGDVLVIPPEFADALKSERQLDFTEVARDDTHGYIPGFEPIGSPFDLVPLVNKYLTRALAKLTKPLWAEASLGVNHVLGTSTEWHPINPGEDIMRIVSRMSSRIFMGEELCKDDDWLKVSIEYTVQLFQTADELRNYPRWTRPYIHWFLPSCQGVRRKLQEARDLLQPHIDRRNAVKKEAIAEGRPSPFDDSIEWFENEYEGKSDPATEQIKLSLVAIHTTTDLLSETMFNIALQPELLGPLREEIVTVLSTEGLKKTSFYNLKLMDSVIKESQRLRPVLLGAFRRMALADVTLPNGDVIKKGTKIICDTTHQWNPEYYPDASKFNAYRFLQMRQTPGQDKRAHLVSTSHDQMGFGHGLHACPGRFFAANEIKIALCHMLLKYDWKLPEGVVPKSKALGMSLLGDREAKLMVKRRAAEIDIDTIGSDE

SEQ ID NO: 34 - DNA, 1280 bp, Artificial Sequence
  synthetic DNA
gtacagaagattaaggcgcgccgcaagccaagcctgcgaagaatgtagtcgagaattgagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgaccactttggatgatactgcttacagatacagaacttctgttccaggtgatgctgaagctattgaagctttggatggatctttcaccactgatactgttttcagagtcactgctactggtgatggattcactttgagagaagttcctgttgatcctcctttgaccaaagtttttcctgatgatgaatctgatgatgaatctgatgctggtgaagatggtgatccagattctagaacttttgttgcttatggtgatgatggtgatttggctggatttgttgttgtttcttattctggatggaacagaagattgactgttgaagatattgaagttgctccagaacatagaggtcatggtgttggaagagctttgatgggattggcaactgagtttgccagagaaagaggtgctggtcatctttggttggaagtcaccaatgtcaatgctccagctattcatgcttacagaagaatgggattcactctttgtggattggatactgctttgtatgatggaactgcttctgatggagaacaagctttgtacatgtccatgccatgtccttaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcggatctgtgagcaaacccgggcatgtgagcaaaagg

SEQ ID NO: 35 - DNA, 807 bp, Artificial Sequence
  synthetic DNA
gaattcatggcccttcgaacgtccctatcacgacccgtaccgcttctggctacacttactgccagcgcaatcggagtatccatattgtctaaaatgatgttttcaacagcaagtgcagagagtccatctccgcaaaaaattttttccggtgcttttgcttccgtaaaactcccgctgcattcaagtgaatacgagtcccatgacacaaagaggcttcgtttcaaacttccgcaagagactgcagtaacgggtttaccgttagcttacttggttcacattccaccgtcccaccatcaaagggacttgactacgccggatgaacctggatacatggacctgttggtaaagaaataccccaaaggccagggctcgacatatctacactccctccagcccggtgatacgttatccttcacatctctacccctcaaaccagcttggaaaacaaacaattttcctcacatcactcttatagctggagggtgtgggatcacgccattattcaacttggctcaagggatacttagagatccggccgaaaaaactaggatgacctttatttttggtgcacgatcagacgaggacgtattactgaaaaaggagttagatggctttgcaaaagagttcccggaaagattcgaggtgaaatatacagcacttttggaagaggtcctagggggcgtgggtcgtgatactaaggtctttgtctgtgggccgaaggagatggaaaaggcacttgtaggaggccgtggcgtattaaaggaaataggcttcgaaaagtctcagatccatactttttgagtcgac

SEQ ID NO: 36 - DNA, 1554 bp, Artificial Sequence
  synthetic DNA
gaattcatggccacggatcttgacctcgtgctgggaaaaagtcagtacgcattattttgtggcataactttatttagctttttcatactaaagtattcccttctcggaaacgggggcaagcaatacccttatatcaaccccaagaaaccctttgagctgtcgaaccagcgagtagtccaggatttcatcgagaacgcacgagacattcttactaagggtcgctcactttacaaggatacgccctacaaggcgcataccgatttaggggacgtcctcgtaatcccgcccgagtttgccgacgctctcaagtccgaaagacagcttgactttaccgaggtcgcgagagacgatactcacggttacattcctggattcgagcccataggttccccgttcgatctggtgccgctcgtcaacaagtatcttacaagggcgttggcaaaactaacaaagccactgtgggccgaagcctcgttaggtgtaaaccatgttctgggcacgtctacggagtggcatcccattaacccaggcgaagatatcatgaggatagtctccagaatgtcatccagaatattcatgggtgaggaactttgtaaagatgacgattggctgaaagtgtcgattgagtacactgtgcagctgtttcaaaccgcagacgaattacgtaactatccacgttggacgcggccctatattcactggttcttgccttcctgtcagggggttcgtcgcaagttgcaggaggcgcgtgatttattgcaaccccatattgataggagaaatgcagtgaagaaagaagcgatcgctgaaggtagaccctcaccattcgacgattcaatagagtggtttgaaaatgagtacgagggcaaatctgatcccgccactgaacaaattaaactatcactggtggcgattcacacaaccacggacctcctgtctgaaaccatgttcaatatagctttgcagccagaactccttggtcccctacgtgaagagatagttacggttctttccacggaaggtctaaaaaagacgtcgttttacaatttgaagttgatggattcggtcataaaggagtcacagcgacttcgacccgttcttctcggtgcgttccgaagaatggcactcgctgacgtaaccttgcccaatggcgacgtaataaagaaagggaccaagatcatttgcgacactacacatcagtggaacccagaatactatcccgatgccagcaagttcaatgcatatcggtttctccaaatgagacagacgcccggtcaggacaaaagagcacaccttgtcagcacaagccacgatcaaatggggttcggacacggcttgcacgcgtgcccaggccggtttttcgcagccaatgagataaagatagcgctgtgtcacatgctattgaagtatgactggaagcttccagaaggtgttgtacctaagtctaaggccctcggcatgtccttactgggggaccgggaagccaaactgatggtcaagaggagagcagccgaaatcgatatagacactattgggagcgatgaatgagtcgac

SEQ ID NO: 37 - DNA, 38 bp, Artificial sequence
  primer
aatttttgaaaattcgaattcatggcccttcgaacgtc

SEQ ID NO: 38 - DNA, 53 bp, Artificial sequence
  primer
ttgtaatccatcgatactagttcaaaaagtatggatctgagacttttcgaagc

SEQ ID NO: 39 - DNA, 39 bp, Artificial sequence
  primer
ctatagggcccgggcgtcgacatggccacggatcttgac

SEQ ID NO: 40 - DNA, 59 bp, Artificial sequence
  primer
gctagccgcggtaccaagctttcattcatcgctcccaatagtgtctatatcgatttcgg